SEQUENCE LISTING (697 sequences)

SEQ ID NO: 1 - protein, 15 aa, Human immunodeficiency virus
EAAEWDRVHPVHAGP

SEQ ID NO: 2 - protein, 9 aa, Homo sapiens
EADPTGHSY

SEQ ID NO: 3 - protein, 17 aa, Homo sapiens
EIKILNIFGVIKGFVEP

SEQ ID NO: 4 - protein, 9 aa, Homo sapiens
EILGFVFTL

SEQ ID NO: 5 - protein, 10 aa, Human immunodeficiency virus
EILKEPVHGV

SEQ ID NO: 6 - protein, 9 aa, Homo sapiens
EIMKWNRER

SEQ ID NO: 7 - protein, 25 aa, Human immunodeficiency virus type 1
EIQKQGQGQWTYQIYQEPFKNLKTG

SEQ ID NO: 8 - protein, 9 aa, Homo sapiens
  Xaa = any amino acid
EIVDXXEKV

SEQ ID NO: 9 - protein, 8 aa, Homo sapiens
EIYKRWII

SEQ ID NO: 10 - protein, 9 aa, Human immunodeficiency virus
EIYKRWIIL

SEQ ID NO: 11 - protein, 21 aa, Homo sapiens
EKAGGAQLGVMQGPMGPMGPR

SEQ ID NO: 12 - protein, 16 aa, Rabies virus
EKDDLSVEAEIAHQIA

SEQ ID NO: 13 - protein, 9 aa, Homo sapiens
EADPTSNTY

SEQ ID NO: 14 - protein, 17 aa, Plasmodium malariae
EKDIAKMEKASSVFNVV

SEQ ID NO: 15 - protein, 14 aa, Streptococcus sp
EKDIQFGREVHAAD

SEQ ID NO: 16 - protein, 17 aa, Streptococcus sp
EKDIQFGREVHAADLLR

SEQ ID NO: 17 - protein, 25 aa, Streptococcus sp
EKDIQFGREVHAADLLRHKQEIAEK

SEQ ID NO: 18 - protein, 14 aa, Human immunodeficiency virus
EKGGLEGLIHSQRR

SEQ ID NO: 19 - protein, 17 aa, Plasmodium malariae
EKGIAKMEKASSVFNVV

SEQ ID NO: 20 - protein, 14 aa, Homo sapiens
EKHKVYACEVTHQG

SEQ ID NO: 21 - protein, 16 aa, Homo sapiens
EKHKVYACEVTHQGLS

SEQ ID NO: 22 - protein, 17 aa, Homo sapiens
EKHKVYACEVTHQGLSS

SEQ ID NO: 23 - protein, 17 aa, Plasmodium malariae
EKKDAKMEKASSVFNVV

SEQ ID NO: 24 - protein, 15 aa, Homo sapiens
EAEQLRAYLDGTGVE

SEQ ID NO: 25 - protein, 17 aa, Plasmodium malariae
EKKGAKMEKASSVFNVV

SEQ ID NO: 26 - protein, 17 aa, Plasmodium malariae
EKKIADMEKASSVFNVV

SEQ ID NO: 27 - protein, 17 aa, Plasmodium malariae
EKKIAFMEKASSVFNVV

SEQ ID NO: 28 - protein, 17 aa, Plasmodium malariae
EKKIAGMEKASSVFNVV

SEQ ID NO: 29 - protein, 17 aa, Plasmodium malariae
EKKIAKDEKASSVFNVV

SEQ ID NO: 30 - protein, 17 aa, Plasmodium malariae
EKKIAKGEKASSVFNVV

SEQ ID NO: 31 - protein, 17 aa, Plasmodium malariae
EKKIAKKEKASSVFNVV

SEQ ID NO: 32 - protein, 17 aa, Plasmodium malariae
EKKIAKMEDASSVFNVV

SEQ ID NO: 33 - protein, 17 aa, Plasmodium malariae
EKKIAKMEFASSVFNVV

SEQ ID NO: 34 - protein, 17 aa, Plasmodium malariae
EKKIAKMEGASSVFNVV

SEQ ID NO: 35 - protein, 13 aa, Mycobacterium leprae
EAFVVEFDLPGIK

SEQ ID NO: 36 - protein, 17 aa, Plasmodium malariae
EKKIAKMEKAESVFNVV

SEQ ID NO: 37 - protein, 17 aa, Plasmodium malariae
EKKIAKMEKAFSVFNVV

SEQ ID NO: 38 - protein, 17 aa, Plasmodium malariae
EKKIAKMEKAKSVFNVV

SEQ ID NO: 39 - protein, 17 aa, Plasmodium malariae
EKKIAKMEKASEVFNVV

SEQ ID NO: 40 - protein, 17 aa, Plasmodium malariae
EKKIAKMEKASFVFNVV

SEQ ID NO: 41 - protein, 17 aa, Plasmodium malariae
EKKIAKMEKASKVFNVV

SEQ ID NO: 42 - protein, 17 aa, Plasmodium malariae
EKKIAKMEKASSDFNVV

SEQ ID NO: 43 - protein, 17 aa, Plasmodium malariae
EKKIAKMEKASSGFNVV

SEQ ID NO: 44 - protein, 17 aa, Plasmodium malariae
EKKIAKMEKASSKFNVV

SEQ ID NO: 45 - protein, 17 aa, Plasmodium malariae
EKKIAKMEKASSVDNVV

SEQ ID NO: 46 - protein, 19 aa, Homo sapiens
EAGAPGLVGPRGERGFPGE

SEQ ID NO: 47 - protein, 17 aa, Plasmodium malariae
EKKIAKMEKASSVFEVV

SEQ ID NO: 48 - protein, 17 aa, Plasmodium malariae
EKKIAKMEKASSVFFVV

SEQ ID NO: 49 - protein, 17 aa, Plasmodium malariae
EKKIAKMEKASSVFKVV

SEQ ID NO: 50 - protein, 17 aa, Plasmodium malariae
EKKIAKMEKASSVFNDV

SEQ ID NO: 51 - protein, 17 aa, Plasmodium malariae
EKKIAKMEKASSVFNGV

SEQ ID NO: 52 - protein, 17 aa, Plasmodium malariae
EKKIAKMEKASSVFNKV

SEQ ID NO: 53 - protein, 17 aa, Plasmodium malariae
EKKIAKMEKASSVFNVV

SEQ ID NO: 54 - protein, 19 aa, Plasmodium malariae
EKKIAKMEKASSVFNVVNS

SEQ ID NO: 55 - protein, 17 aa, Plasmodium malariae
EKKIAKMEKASSVGNVV

SEQ ID NO: 56 - protein, 17 aa, Plasmodium malariae
EKKIAKMEKASSVKNVV

SEQ ID NO: 57 - protein, 20 aa, Homo sapiens
EAGHQKVVFYILIQRKPLFY

SEQ ID NO: 58 - protein, 17 aa, Plasmodium malariae
EKKIAKMEKDSSVFNVV

SEQ ID NO: 59 - protein, 17 aa, Plasmodium malariae
EKKIAKMEKESSVFNVV

SEQ ID NO: 60 - protein, 17 aa, Plasmodium malariae
EKKIAKMEKVSSVFNVV

SEQ ID NO: 61 - protein, 17 aa, Plasmodium malariae
EKKIAKMEKYSSVFNVV

SEQ ID NO: 62 - protein, 17 aa, Plasmodium malariae
EKKIAKMFKASSVFNVV

SEQ ID NO: 63 - protein, 17 aa, Plasmodium malariae
EKKIAKMGKASSVFNVV

SEQ ID NO: 64 - protein, 17 aa, Plasmodium malariae
EKKIAKMKKASSVFNVV

SEQ ID NO: 65 - protein, 17 aa, Plasmodium malariae
EKKIIKMEKASSVFNVV

SEQ ID NO: 66 - protein, 17 aa, Plasmodium malariae
EKKIKKMEKASSVFNVV

SEQ ID NO: 67 - protein, 17 aa, Plasmodium malariae
EKKIVKMEKASSVFNVV

SEQ ID NO: 68 - protein, 11 aa, Homo sapiens
EAIIHVLHSRH

SEQ ID NO: 69 - protein, 17 aa, Plasmodium malariae
EKKIYKMEKASSVFNVV

SEQ ID NO: 70 - protein, 17 aa, Plasmodium malariae
EKKKAKMEKASSVFNVV

SEQ ID NO: 71 - protein, 13 aa, Homo sapiens
EKKYFAATQFEPL

SEQ ID NO: 72 - protein, 17 aa, Homo sapiens [sequence omitted]

SEQ ID NO: 73 - protein, 13 aa, Streptococcus sp
EKLAKQAEELAKL

SEQ ID NO: 74 - protein, 21 aa, Streptococcus sp
EKQISDASRQGLRRDLDASRE

SEQ ID NO: 75 - protein, 17 aa, Plasmodium malariae
EKYIAKMEKASSVFNVV

SEQ ID NO: 76 - protein, 11 aa, Influenza virus
EKYVKQNTLKA

SEQ ID NO: 77 - protein, 13 aa, Homo sapiens
EKYVKQNTLKLAT

SEQ ID NO: 78 - protein, 15 aa, Homo sapiens
ELAAAMKRHGLDNYR

SEQ ID NO: 79 - protein, 12 aa, Homo sapiens
EAIQPGCIGGPK

SEQ ID NO: 80 - protein, 10 aa, Human immunodeficiency virus
ELAENREILK

SEQ ID NO: 81 - protein, 10 aa, Homo sapiens
ELAGIGILTV

SEQ ID NO: 82 - protein, 15 aa, Homo sapiens
ELAQYLDLVRALEAA

SEQ ID NO: 83 - protein, 9 aa, Plasmodium falciparum
ELDVLKKLV

SEQ ID NO: 84 - protein, 15 aa, Plasmodium falciparum
ELDYANDIEKKICKM

SEQ ID NO: 85 - protein, 15 aa, Homo sapiens
ELFRKDIAAKYKEGY

SEQ ID NO: 86 - protein, 18 aa, Homo sapiens
ELFRKDIAAKYKELGYGK

SEQ ID NO: 87 - protein, 12 aa, Homo sapiens
ELGGWKLKLQSD

SEQ ID NO: 88 - protein, 9 aa, Clostridium tetani
ELIHVLHGL

SEQ ID NO: 89 - protein, 9 aa, Newcastle disease virus
ELIHVNHLI

SEQ ID NO: 90 - protein, 13 aa, Homo sapiens
EAIYDICRRNLDI

SEQ ID NO: 91 - protein, 9 aa, Homo sapiens
ELIRVEGNL

SEQ ID NO: 92 - protein, 9 aa, Mus musculus
ELIRVVHQL

SEQ ID NO: 93 - protein, 8 aa, Homo sapiens
ELKEKTQL

SEQ ID NO: 94 - protein, 9 aa, Homo sapiens
  Xaa = any amino acid
ELKEKXYEL

SEQ ID NO: 95 - protein, 9 aa, Homo sapiens
  Xaa = any amino acid
ELKIKVYXL

SEQ ID NO: 96 - protein, 8 aa, Homo sapiens
ELKKKTNL

SEQ ID NO: 97 - protein, 6 aa, Homo sapiens
ELKLKG

SEQ ID NO: 98 - protein, 9 aa, Influenza A virus
ELKSKYWAI

SEQ ID NO: 99 - protein, 9 aa, Influenza virus
ELKSRYWAI

SEQ ID NO: 100 - protein, 9 aa, Homo sapiens
ELKVKNLEL

SEQ ID NO: 101 - protein, 17 aa, Homo sapiens
EAIYDICRRNLDIERPT

SEQ ID NO: 102 - protein, 12 aa, Human immunodeficiency virus
ELLGIWGCSGKL

SEQ ID NO: 103 - protein, 9 aa, Homo sapiens
ELNEALELK

SEQ ID NO: 104 - protein, 9 aa, Epstein Barr virus
ELRGRAYGL

SEQ ID NO: 105 - protein, 9 aa, Human immunodeficiency virus
ELRSLYNTV

SEQ ID NO: 106 - protein, 9 aa, Influenza virus
ELRSRYWAI

SEQ ID NO: 107 - protein, 9 aa, Homo sapiens
  Xaa = any amino acid
ELVDXXEKV

SEQ ID NO: 108 - protein, 9 aa, Homo sapiens
ELVHFLLLK

SEQ ID NO: 109 - protein, 10 aa, Human immunodeficiency virus
ELVNQIIEQL

SEQ ID NO: 110 - protein, 9 aa, Homo sapiens
ELVSEFSRM

SEQ ID NO: 111 - protein, 9 aa, Homo sapiens
ELVSEFSRV

SEQ ID NO: 112 - protein, 10 aa, Homo sapiens
EAAGIGILTV

SEQ ID NO: 113 - protein, 17 aa, Homo sapiens
EAKPGKAGERGPPGPQG

SEQ ID NO: 114 - protein, 9 aa, Homo sapiens
ELVSEVSKV

SEQ ID NO: 115 - protein, 19 aa, Human immunodeficiency virus
ELYPLTSLRSLFGNDPSSQ

SEQ ID NO: 116 - protein, 9 aa, Homo sapiens
EMFRELNEA

SEQ ID NO: 117 - protein, 8 aa, Homo sapiens
ENAAFVLL

SEQ ID NO: 118 - protein, 15 aa, Homo sapiens
ENAVVHFFKNIVTPR

SEQ ID NO: 119 - protein, 15 aa, Plasmodium falciparum
ENDIEKKICKMEKCS

SEQ ID NO: 120 - protein, 23 aa, Homo sapiens
ENGEWAIQHRPAKMLLDPAAPAQ

SEQ ID NO: 121 - protein, 12 aa, Homo sapiens
ENIEFLEDTDMK

SEQ ID NO: 122 - protein, 18 aa, Homo sapiens
ENIEFLEDTDMKSLENKS

SEQ ID NO: 123 - protein, 8 aa, Homo sapiens
ENIFYCPI

SEQ ID NO: 124 - protein, 13 aa, Streptococcus sp
EALAKQAEELAKL

SEQ ID NO: 125 - protein, 15 aa, Homo sapiens
ENPAVHFFKNIVTPR

SEQ ID NO: 126 - protein, 15 aa, Homo sapiens
ENPVAHFFKNIVTPR

SEQ ID NO: 127 - protein, 15 aa, Homo sapiens
ENPVKHFFKNIVTPR

SEQ ID NO: 128 - protein, 15 aa, Homo sapiens
ENPVVAFFKNIVTPR

SEQ ID NO: 129 - protein, 15 aa, Homo sapiens
ENPVVDFFKNIVTPR

SEQ ID NO: 130 - protein, 15 aa, Homo sapiens
ENPVVHAFKNIVTPR

SEQ ID NO: 131 - protein, 15 aa, Homo sapiens
ENPVVHFAKNIVTPR

SEQ ID NO: 132 - protein, 15 aa, Homo sapiens
ENPVVHFFANIVTPR

SEQ ID NO: 133 - protein, 15 aa, Homo sapiens
ENPVVHFFKAIVTPR

SEQ ID NO: 134 - protein, 15 aa, Homo sapiens
ENPVVHFFKNAVTPR

SEQ ID NO: 135 - protein, 15 aa, Homo sapiens
EALIHQLKINPYVLS

SEQ ID NO: 136 - protein, 15 aa, Homo sapiens
ENPVVHFFKNIATPR

SEQ ID NO: 137 - protein, 15 aa, Homo sapiens
ENPVVHFFKNIATPR

SEQ ID NO: 138 - protein, 15 aa, Homo sapiens
ENPVVHFFKNIVAPR

SEQ ID NO: 139 - protein, 14 aa, Homo sapiens
ENPVVHFFKNIVTA

SEQ ID NO: 140 - protein, 15 aa, Homo sapiens
ENPVVHFFKNIVTAR

SEQ ID NO: 141 - protein, 15 aa, Homo sapiens
ENPVVHFFKNIVTPA

SEQ ID NO: 142 - protein, 15 aa, Homo sapiens
ENPVVHFFKNIVTPR

SEQ ID NO: 143 - protein, 19 aa, Homo sapiens
ENPVVHFFKNIVTPRTPPY

SEQ ID NO: 144 - protein, 15 aa, Homo sapiens
ENPVVHFFRNIVTPR

SEQ ID NO: 145 - protein, 15 aa, Homo sapiens
ENPVVHYFKNIVTPR

SEQ ID NO: 146 - protein, 12 aa, Homo sapiens
EALVRQGLAKVA

SEQ ID NO: 147 - protein, 15 aa, Homo sapiens
ENPVVKFFKNIVTPR

SEQ ID NO: 148 - protein, 9 aa, Plasmodium falciparum
EPAPFDETL

SEQ ID NO: 149 - protein, 14 aa, Homo sapiens
EPDHYVVVGAQRDA

SEQ ID NO: 150 - protein, 20 aa, Homo sapiens
EPEASPSLWEIEFAKQLASV

SEQ ID NO: 151 - protein, 14 aa, Homo sapiens
EPEITILNVKLQPA

SEQ ID NO: 152 - protein, 9 aa, Human immunodeficiency virus
EPEPHILLF

SEQ ID NO: 153 - protein, 11 aa, Human immunodeficiency virus
EPFKNLKTGKY

SEQ ID NO: 154 - protein, 16 aa, Homo sapiens
EPFLYILGKSRVLEAQ

SEQ ID NO: 155 - protein, 11 aa, Human immunodeficiency virus
EPFRDYVDRFY

SEQ ID NO: 156 - protein, 9 aa, Homo sapiens
EPGPVTAQV

SEQ ID NO: 157 - protein, 20 aa, Staphylococcus sp
EALVRQGLAKVAYVYKPNNT

SEQ ID NO: 158 - protein, 10 aa, Homo sapiens
EPIDKEIYPL

SEQ ID NO: 159 - protein, 8 aa, Human immunodeficiency virus
EPIDKELY

SEQ ID NO: 160 - protein, 9 aa, Homo sapiens
EPILRSLAY

SEQ ID NO: 161 - protein, 9 aa, Human immunodeficiency virus
EPIVGAETF

SEQ ID NO: 162 - protein, 10 aa, Human immunodeficiency virus
EPIVGAETFY

SEQ ID NO: 163 - protein, 9 aa, Homo sapiens
EPIVGAETI

SEQ ID NO: 164 - protein, 17 aa, Homo sapiens
EPKDFVYALNLTQTLNP

SEQ ID NO: 165 - protein, 26 aa, Homo sapiens
EPKSQDIYLRLLVKLYRFLARRTNST

SEQ ID NO: 166 - protein, 8 aa, Homo sapiens
EPKYKTQL

SEQ ID NO: 167 - protein, 9 aa, Homo sapiens
EPLDLPQII

SEQ ID NO: 168 - protein, 20 aa, Homo sapiens
EALVRQGLARVAYVYKPNNT

SEQ ID NO: 169 - protein, 17 aa, Mus musculus
EPLVPLDNHIPENAQPG

SEQ ID NO: 170 - protein, 9 aa, Human immunodeficiency virus
EPPFLWMGY

SEQ ID NO: 171 - protein, 15 aa, Homo sapiens
EPRAPWIEQEGPEYW

SEQ ID NO: 172 - protein, 17 aa, Plasmodium falciparum
EPSDKHIEQYLKKIKNS

SEQ ID NO: 173 - protein, 8 aa, Human immunodeficiency virus
EPVHEVYY

SEQ ID NO: 174 - protein, 10 aa, Homo sapiens
EPVPLQLPPL

SEQ ID NO: 175 - protein, 10 aa, Human papillomavirus
EPYGDSLFFY

SEQ ID NO: 176 - protein, 10 aa, Homo sapiens
EQARAAVDTY

SEQ ID NO: 177 - protein, 11 aa, Sus scrofa
EQCCTSICSLY

SEQ ID NO: 178 - protein, 17 aa, Homo sapiens
EQDFLTKHASHTGSWIG

SEQ ID NO: 179 - protein, 20 aa, Staphylococcus aureus
EALVRQGLARVAYVYRPNNT

SEQ ID NO: 180 - protein, 31 aa, Clostridium tetani
EQDPSGATTKSAMLTNLIIFGPGPVLNKNEV

SEQ ID NO: 181 - protein, 13 aa, Streptococcus sp
EQLAKQAEELAKL

SEQ ID NO: 182 - protein, 20 aa, Streptococcus sp
EQLAKQAEELAKLRAGKASD

SEQ ID NO: 183 - protein, 16 aa, Human papillomavirus
EQMFVRHLFNRAGTVG

SEQ ID NO: 184 - protein, 15 aa, Human immunodeficiency virus
EQMHEDIISLWDQSL

SEQ ID NO: 185 - protein, 15 aa, Homo sapiens
EQNQEQRRAAQRAAG

SEQ ID NO: 186 - protein, 15 aa, Human immunodeficiency virus
EQRGPGRAFVTIGKI

SEQ ID NO: 187 - protein, 14 aa, Homo sapiens
EQSLITVEGDKASM

SEQ ID NO: 188 - protein, 16 aa, Mus musculus
EQTQQIRLQAEIFQAR

SEQ ID NO: 189 - protein, 21 aa, Influenza virus
EQTSLYVQASGRVTVSTRRSQ

SEQ ID NO: 190 - protein, 9 aa, Homo sapiens
EAPGNYPAL

SEQ ID NO: 191 - protein, 10 aa, Plasmodium falciparum
EQYLKKIKNS

SEQ ID NO: 192 - protein, 15 aa, Homo sapiens
ERADLIAYLKQATAK

SEQ ID NO: 193 - protein, 9 aa, Homo sapiens
ERAKIRGSL

SEQ ID NO: 194 - protein, 20 aa, Homo sapiens
EREEALTTNVWIEMQWCDYR

SEQ ID NO: 195 - protein, 9 aa, Influenza virus
ERELVRKTR

SEQ ID NO: 196 - protein, 16 aa, Human immunodeficiency virus
ERFAVNPGLLETSEGC

SEQ ID NO: 197 - protein, 8 aa, Homo sapiens
  Xaa = any amino acid
ERFTXIXG

SEQ ID NO: 198 - protein, 16 aa, Homo sapiens
ERGPPGPQGARGFPGT

SEQ ID NO: 199 - protein, 25 aa, Mus musculus
ERITQIAKGQEQWFRVNLRTLLGYY

SEQ ID NO: 200 - protein, 9 aa, Homo sapiens
ERLAIRGSL

SEQ ID NO: 201 - protein, 15 aa, Homo sapiens
EAPVVHFFKNIVTPR

SEQ ID NO: 202 - protein, 14 aa, Mycobacterium leprae
ERLAKLAGGVAVIK

SEQ ID NO: 203 - protein, 28 aa, Mycobacterium leprae
ERLAKLAGGVAVIKAGAATEVELKERKH

SEQ ID NO: 204 - protein, 9 aa, Homo sapiens
ERLKARGSL

SEQ ID NO: 205 - protein, 9 aa, Homo sapiens
ERLKIAGSL

SEQ ID NO: 206 - protein, 9 aa, Homo sapiens
ERLKIRASL

SEQ ID NO: 207 - protein, 9 aa, Homo sapiens
ERLKIRGAL

SEQ ID NO: 208 - protein, 9 aa, Homo sapiens
ERLKIRGSA

SEQ ID NO: 209 - protein, 9 aa, Homo sapiens
ERLKIRGSL

SEQ ID NO: 210 - protein, 16 aa, Drosophila
ERLNSQDQQEDSSLVE

SEQ ID NO: 211 - protein, 18 aa, Homo sapiens
ERPTYTNLNRLIGQIVSS

SEQ ID NO: 212 - protein, 11 aa, Homo sapiens
EAVHAAHAEIN

SEQ ID NO: 213 - protein, 9 aa, Homo sapiens
ERTLHLVEL

SEQ ID NO: 214 - protein, 9 aa, Human immunodeficiency virus
ERYLKDQQL

SEQ ID NO: 215 - protein, 9 aa, Homo sapiens
ERYLKDQQL

SEQ ID NO: 216 - protein, 10 aa, Homo sapiens
ERYLKDQQLL

SEQ ID NO: 217 - protein, 10 aa, Human immunodeficiency virus
ERYLKDQQLL

SEQ ID NO: 218 - protein, 11 aa, Human immunodeficiency virus
ERYLKDQQLLG

SEQ ID NO: 219 - protein, 11 aa, Homo sapiens
ERYLKDQQLLG

SEQ ID NO: 220 - protein, 13 aa, Human immunodeficiency virus
ERYLKDQQLLGIW

SEQ ID NO: 221 - protein, 13 aa, Homo sapiens
ERYLKDQQLLGIW

SEQ ID NO: 222 - protein, 22 aa, Human immunodeficiency virus
ERYLKDQQLLGIWGCSGKLICG

SEQ ID NO: 223 - protein, 10 aa, Homo sapiens
EAAGTGILTV

SEQ ID NO: 224 - protein, 8 aa, Homo sapiens
EAYLGKKV

SEQ ID NO: 225 - protein, 11 aa, Homo sapiens
ERYLRDQQLLG

SEQ ID NO: 226 - protein, 9 aa, Homo sapiens
ERYPRYNQL

SEQ ID NO: 227 - protein, 9 aa, Homo sapiens
ERYQKSTEL

SEQ ID NO: 228 - protein, 11 aa, Homo sapiens
  Xaa = any amino acid
ESFLXYKKGIY

SEQ ID NO: 229 - protein, 15 aa, Human immunodeficiency virus
ESFRSGVETTTPPQK

SEQ ID NO: 230 - protein, 9 aa, Homo sapiens
ESGPSIVHR

SEQ ID NO: 231 - protein, 10 aa, Homo sapiens
ESGPSIVHRK

SEQ ID NO: 232 - protein, 10 aa, Homo sapiens
ESLFRAVITK

SEQ ID NO: 233 - protein, 11 aa, Gallus gallus
ESNFNTQATNR

SEQ ID NO: 234 - protein, 12 aa, Influenza A virus
ESTGNLIAPEYG

SEQ ID NO: 235 - protein, 18 aa, Homo sapiens
  Xaa = any amino acid
EDENLYEGLNLDDXSMYE

SEQ ID NO: 236 - protein, 6 aa, Human immunodeficiency virus
ESVQIN

SEQ ID NO: 237 - protein, 20 aa, Homo sapiens
ESWGAVWRIDTPDKLTGPFT

SEQ ID NO: 238 - protein, 11 aa, Homo sapiens
ETDIIIDRSEY

SEQ ID NO: 239 - protein, 11 aa, Homo sapiens
ETDIILDRSEY

SEQ ID NO: 240 - protein, 11 aa, Homo sapiens
ETDILIDRSEY

SEQ ID NO: 241 - protein, 11 aa, Homo sapiens
ETDILLDRSEY

SEQ ID NO: 242 - protein, 11 aa, Homo sapiens
ETDLIIDRSEY

SEQ ID NO: 243 - protein, 11 aa, Homo sapiens
ETDLILDRSEY

SEQ ID NO: 244 - protein, 11 aa, Homo sapiens
ETDLLIDRSEY

SEQ ID NO: 245 - protein, 11 aa, Homo sapiens
ETDLLLDRSEY

SEQ ID NO: 246 - protein, 17 aa, Plasmodium malariae
EDKIAKMEKASSVFNVV

SEQ ID NO: 247 - protein, 11 aa, Homo sapiens
  Xaa = any amino acid
ETDXXXDRSEY

SEQ ID NO: 248 - protein, 10 aa, Homo sapiens
ETFNTPAHYV

SEQ ID NO: 249 - protein, 11 aa, Human immunodeficiency virus
ETFYVDGAANR

SEQ ID NO: 250 - protein, 9 aa, Homo sapiens
ETIIPDWSY

SEQ ID NO: 251 - protein, 9 aa, Homo sapiens
ETILPDWSY

SEQ ID NO: 252 - protein, 10 aa, Human immunodeficiency virus
ETINEEAAEW

SEQ ID NO: 253 - protein, 13 aa, Streptococcus sp
ETLAKQAEELAKL

SEQ ID NO: 254 - protein, 9 aa, Homo sapiens
ETLIPDWSY

SEQ ID NO: 255 - protein, 9 aa, Homo sapiens
ETLLPDWSY

SEQ ID NO: 256 - protein, 12 aa, Homo sapiens
ETLLRAVESYLL

SEQ ID NO: 257 - protein, 9 aa, Influenza virus
EDLRVLSFI

SEQ ID NO: 258 - protein, 14 aa, Homo sapiens
ETLLRAVESYLLAH

SEQ ID NO: 259 - protein, 16 aa, Homo sapiens
ETLLRAVESYLLAHSD

SEQ ID NO: 260 - protein, 8 aa, Human papillomavirus
ETTDLYCY

SEQ ID NO: 261 - protein, 13 aa, Homo sapiens
ETTEESLRNYYEG

SEQ ID NO: 262 - protein, 13 aa, Homo sapiens
ETTEESLRNYYEQ

SEQ ID NO: 263 - protein, 11 aa, Homo sapiens
ETVAVGVIKAV

SEQ ID NO: 264 - protein, 9 aa, Homo sapiens
  Xaa = any amino acid
ETXXPDWSY

SEQ ID NO: 265 - protein, 16 aa, Homo sapiens
ETYGEMADCCAKQEPE

SEQ ID NO: 266 - protein, 11 aa, Human immunodeficiency virus
ETYYVNGAANR

SEQ ID NO: 267 - protein, 20 aa, Homo sapiens
EVALCLPRSELLFQQWQRQG

SEQ ID NO: 268 - protein, 11 aa, Plasmodium yoelii
EDSYVPSAEQI

SEQ ID NO: 269 - protein, 9 aa, Homo sapiens
EVAPPEYHR

SEQ ID NO: 270 - protein, 10 aa, Homo sapiens
EVAPPEYHRK

SEQ ID NO: 271 - protein, 9 aa, Homo sapiens
EVAPPLLFV

SEQ ID NO: 272 - protein, 13 aa, Schistosoma mansoni
EVCVRQLKAIANK

SEQ ID NO: 273 - protein, 9 aa, Homo sapiens
EVDPIGHLY

SEQ ID NO: 274 - protein, 9 aa, Homo sapiens
EVDPIGHSY

SEQ ID NO: 275 - protein, 9 aa, Influenza virus
EVDPIGHVY

SEQ ID NO: 276 - protein, 9 aa, Homo sapiens
EVDPTSNTY

SEQ ID NO: 277 - protein, 10 aa, Homo sapiens
EVILIDPFHK

SEQ ID NO: 278 - protein, 9 aa, Human immunodeficiency virus
EVIPMFSAL

SEQ ID NO: 279 - protein, 17 aa, Homo sapiens
EDVIPEGWKADTSYSAK

SEQ ID NO: 280 - protein, 17 aa, Plasmodium malariae
EVKIAKMEKASSVFNVV

SEQ ID NO: 281 - protein, 11 aa, Human immunodeficiency virus
EVLVWRFDSKL

SEQ ID NO: 282 - protein, 15 aa, Human immunodeficiency virus
EVVIRSANFTDNAKT

SEQ ID NO: 283 - protein, 9 aa, Homo sapiens
EVVPISHLY

SEQ ID NO: 284 - protein, 11 aa, Homo sapiens
EVWREEAYHAA

SEQ ID NO: 285 - protein, 15 aa, Homo sapiens
EVWREEAYHAADIKD

SEQ ID NO: 286 - protein, 11 aa, Rubella virus
EVWVTPVIGSA

SEQ ID NO: 287 - protein, 18 aa, Rubella virus
EVWVTPVIGSARKCGLHI

SEQ ID NO: 288 - protein, 12 aa, Rubella virus
EVWVTPVIGSQA

SEQ ID NO: 289 - protein, 12 aa, Rubella virus
EVWVTPVIGTQA

SEQ ID NO: 290 - protein, 16 aa, Homo sapiens
EEDFHVDQATTVKVPM

SEQ ID NO: 291 - protein, 10 aa, Human papillomavirus
EVYDFAFRDL

SEQ ID NO: 292 - protein, 25 aa, Human immunodeficiency virus
EWRFDSRLAFHHVAREHPEYFNKNK

SEQ ID NO: 293 - protein, 17 aa, Human immunodeficiency virus
EWRFDSRLAFHHVAREL

SEQ ID NO: 294 - protein, 9 aa, Homo sapiens
EWTSSNVME

SEQ ID NO: 295 - protein, 10 aa, Homo sapiens
EWTSSNVMEE

SEQ ID NO: 296 - protein, 9 aa, Homo sapiens
EWVSLFRMQ

SEQ ID NO: 297 - protein, 9 aa, Homo sapiens
EWWGLGRWR

SEQ ID NO: 298 - protein, 9 aa, respiratory syncytial virus
EYALGVVGV

SEQ ID NO: 299 - protein, 17 aa, Homo sapiens
EYILYNKGIMGEDSYPY

SEQ ID NO: 300 - protein, 9 aa, Homo sapiens
EYIVLLFLL

SEQ ID NO: 301 - protein, 16 aa, Homo sapiens
EEDFHVDQVTTVKVPM

SEQ ID NO: 302 - protein, 18 aa, Homo sapiens
EYKLVVVGAAGVGKSALT

SEQ ID NO: 303 - protein, 18 aa, Homo sapiens
EYKLVVVGADGVGKSALT

SEQ ID NO: 304 - protein, 18 aa, Homo sapiens
EYKLVVVGAGDVGKSALT

SEQ ID NO: 305 - protein, 18 aa, Homo sapiens
EYKLVVVGARGVGKSALT

SEQ ID NO: 306 - protein, 18 aa, Homo sapiens
EYKLVVVGASGVGKSALT

SEQ ID NO: 307 - protein, 10 aa, Mus musculus
EYKLVVVGAV

SEQ ID NO: 308 - protein, 18 aa, Homo sapiens
EYKLVVVGAVGVGKSALT

SEQ ID NO: 309 - protein, 15 aa, Homo sapiens
EYLENPKKYIPGTKM

SEQ ID NO: 310 - protein, 15 aa, Mus musculus
EYLINVIHAFQYVIG

SEQ ID NO: 311 - protein, 10 aa, Plasmodium falciparum
EYLNKIQNSL

SEQ ID NO: 312 - protein, 8 aa, Homo sapiens
EEDPVKKV

SEQ ID NO: 313 - protein, 9 aa, Human papillomavirus
EYRHYCYSL

SEQ ID NO: 314 - protein, 9 aa, Human T-cell lymphotropic virus
EYTNIPISL

SEQ ID NO: 315 - protein, 9 aa, Homo sapiens
EYVLLLFLL

SEQ ID NO: 316 - protein, 9 aa, Homo sapiens
EYVNARHCL

SEQ ID NO: 317 - protein, 16 aa, Homo sapiens
EYVRFDSFVGEYRAVT

SEQ ID NO: 318 - protein, 14 aa, Homo sapiens
EYWDGETRKVKAQS

SEQ ID NO: 319 - protein, 14 aa, Homo sapiens
EYWDGETRNMKASA

SEQ ID NO: 320 - protein, 11 aa, Homo sapiens
EYWQATWIPEW

SEQ ID NO: 321 - protein, 20 aa, Homo sapiens
QAAPAIQACVEACNLIACAR

SEQ ID NO: 322 - protein, 11 aa, Homo sapiens
QADHAAHAEIN

SEQ ID NO: 323 - protein, 20 aa, Human immunodeficiency virus
EEEEVGFPVTPQVPLRPMTY

SEQ ID NO: 324 - protein, 9 aa, Human papillomavirus type 16
QAEPDRAHY

SEQ ID NO: 325 - protein, 10 aa, Hepatitis B virus
QAFTFSPTYK

SEQ ID NO: 326 - protein, 10 aa, Mus musculus
QAHRALDLVA

SEQ ID NO: 327 - protein, 20 aa, Homo sapiens
QAHSLERVCHCLGKWLGHPD

SEQ ID NO: 328 - protein, 11 aa, Homo sapiens
QAIHAAHAEIN

SEQ ID NO: 329 - protein, 14 aa, Homo sapiens
QAIKANSKFIGITE

SEQ ID NO: 330 - protein, 6 aa, Human immunodeficiency virus
QAISPR

SEQ ID NO: 331 - protein, 11 aa, Human immunodeficiency virus
QAISPRTLNAW

SEQ ID NO: 332 - protein, 19 aa, Homo sapiens
QAKFFACIKRSDGSCAWYR

SEQ ID NO: 333 - protein, 28 aa, Homo sapiens
QAKFFACIKRSDGSCAWYRGAAPPKQEF

SEQ ID NO: 334 - protein, 9 aa, Homo sapiens
EADATGHSY

SEQ ID NO: 335 - protein, 8 aa, Homo sapiens
EEEPVKKI

SEQ ID NO: 336 - protein, 24 aa, Streptococcus sp
QAKKATEAELNNLKAELAKVTEQK

SEQ ID NO: 337 - protein, 9 aa, Epstein Barr virus
QAKWRLQTL

SEQ ID NO: 338 - protein, 11 aa, Homo sapiens
QALHAAHAEIN

SEQ ID NO: 339 - protein, 7 aa, Equus sp
QAPGFTY

SEQ ID NO: 340 - protein, 9 aa, Homo sapiens
QAPGNYPAL

SEQ ID NO: 341 - protein, 11 aa, Homo sapiens
QARHAAHAEIN

SEQ ID NO: 342 - protein, 14 aa, Homo sapiens
QARILAVERYLKDQ

SEQ ID NO: 343 - protein, 9 aa, Human immunodeficiency virus
QASQEVKNW

SEQ ID NO: 344 - protein, 10 aa, Homo sapiens
QASQEVKNWM

SEQ ID NO: 345 - protein, 13 aa, Homo sapiens
QASQEVKNWMTET

SEQ ID NO: 346 - protein, 11 aa, Human immunodeficiency virus
EEEVGFPVRPQ

SEQ ID NO: 347 - protein, 11 aa, Homo sapiens
QATHAAHAEIN

SEQ ID NO: 348 - protein, 28 aa, Homo sapiens
QATNRNTDGSTDYGILQINSRWWCNDGR

SEQ ID NO: 349 - protein, 11 aa, Homo sapiens
QAVEAAHAEIN

SEQ ID NO: 350 - protein, 11 aa, Homo sapiens
QAVHAAEAEIN

SEQ ID NO: 351 - protein, 11 aa, Homo sapiens
QAVHAAHADIN

SEQ ID NO: 352 - protein, 11 aa, Homo sapiens
QAVHAAHAEDN

SEQ ID NO: 353 - protein, 11 aa, Homo sapiens
QAVHAAHAEID

SEQ ID NO: 354 - protein, 11 aa, Homo sapiens
QAVHAAHAEII

SEQ ID NO: 355 - protein, 11 aa, Homo sapiens
QAVHAAHAEIN

SEQ ID NO: 356 - protein, 15 aa, Homo sapiens
QAVHAAHAEINEAGR

SEQ ID NO: 357 - protein, 13 aa, Mycobacterium leprae
EEFAVEFDLPGIK

SEQ ID NO: 358 - protein, 11 aa, Homo sapiens
QAVHAAHAEIQ

SEQ ID NO: 359 - protein, 11 aa, Homo sapiens
QAVHAAHAEIT

SEQ ID NO: 360 - protein, 11 aa, Homo sapiens
QAVHAAHAEIY

SEQ ID NO: 361 - protein, 11 aa, Homo sapiens
QAVHAAHAELN

SEQ ID NO: 362 - protein, 11 aa, Homo sapiens
QAVHAAHAERN

SEQ ID NO: 363 - protein, 11 aa, Homo sapiens
QAVHAAHAETN

SEQ ID NO: 364 - protein, 11 aa, Homo sapiens
QAVHAAHAEYN

SEQ ID NO: 365 - protein, 11 aa, Homo sapiens
QAVHAAHAIIN

SEQ ID NO: 366 - protein, 11 aa, Homo sapiens
QAVHAAHAQIN

SEQ ID NO: 367 - protein, 11 aa, Homo sapiens
QAVHAAHARIN

SEQ ID NO: 368 - protein, 9 aa, Homo sapiens
EEFQFIKKA

SEQ ID NO: 369 - protein, 11 aa, Homo sapiens
QAVHAAHAYIN

SEQ ID NO: 370 - protein, 11 aa, Homo sapiens
QAVHAAHGEIN

SEQ ID NO: 371 - protein, 11 aa, Homo sapiens
QAVHAAHSEIN

SEQ ID NO: 372 - protein, 11 aa, Homo sapiens
QAVHAAHVEIN

SEQ ID NO: 373 - protein, 11 aa, Homo sapiens
QAVHAAKAEIN

SEQ ID NO: 374 - protein, 11 aa, Homo sapiens
QAVHAALAEIN

SEQ ID NO: 375 - protein, 11 aa, Homo sapiens
QAVHAAQAEIN

SEQ ID NO: 376 - protein, 11 aa, Homo sapiens
QAVHAARAEIN

SEQ ID NO: 377 - protein, 11 aa, Homo sapiens
QAVHAGHAEIN

SEQ ID NO: 378 - protein, 11 aa, Homo sapiens
QAVHARHAEIN

SEQ ID NO: 379 - protein, 13 aa, Mycobacterium leprae
EEFVAEFDLPGIK

SEQ ID NO: 380 - protein, 11 aa, Homo sapiens
QAVHASHAEIN

SEQ ID NO: 381 - protein, 11 aa, Homo sapiens
QAVHAVHAEIN

SEQ ID NO: 382 - protein, 11 aa, Homo sapiens
QAVHAYHAEIN

SEQ ID NO: 383 - protein, 11 aa, Homo sapiens
QAVHGAHAEIN

SEQ ID NO: 384 - protein, 11 aa, Homo sapiens
QAVHSAHAEIN

SEQ ID NO: 385 - protein, 11 aa, Homo sapiens
QAVHVAHAEIN

SEQ ID NO: 386 - protein, 11 aa, Homo sapiens
QAVHYAHAEIN

SEQ ID NO: 387 - protein, 11 aa, Homo sapiens
QAVKAAHAEIN

SEQ ID NO: 388 - protein, 11 aa, Homo sapiens
QAVLAAHAEIN

SEQ ID NO: 389 - protein, 11 aa, Homo sapiens
QAVQAAHAEIN

SEQ ID NO: 390 - protein, 13 aa, Mycobacterium leprae
EEFVVAFDLPGIK

SEQ ID NO: 391 - protein, 11 aa, Homo sapiens
QAVRAAHAEIN

SEQ ID NO: 392 - protein, 16 aa, Homo sapiens
QDFLTKHASHTGSWIG

SEQ ID NO: 393 - protein, 15 aa, Homo sapiens
QDILIRLFKSHPETL

SEQ ID NO: 394 - protein, 13 aa, Homo sapiens
QDLELSWNLNGLQ

SEQ ID NO: 395 - protein, 14 aa, Homo sapiens
QDLELSWNLNGLQA

SEQ ID NO: 396 - protein, 16 aa, Homo sapiens
QDLELSWNLNGLQADL

SEQ ID NO: 397 - protein, 15 aa, Homo sapiens
QDVDYFRHPPEVSLL

SEQ ID NO: 398 - protein, 15 aa, Mus musculus
QDYEYLINVIHAFQY

SEQ ID NO: 399 - protein, 9 aa, Homo sapiens
QEEEGPSTF

SEQ ID NO: 400 - protein, 14 aa, Clostridium tetani
QEIKANSKFIGITE

SEQ ID NO: 401 - protein, 13 aa, Mycobacterium leprae
EEFVVEADLPGIK

SEQ ID NO: 402 - protein, 12 aa, Clostridium tetani
QEIYMQHTYPIS

SEQ ID NO: 403 - protein, 17 aa, Homo sapiens
QELKNKYYQVPRKGIQA

SEQ ID NO: 404 - protein, 8 aa, Influenza virus
QESTGNLI

SEQ ID NO: 405 - protein, 13 aa, Streptococcus sp
QFGKEVHAADLLR

SEQ ID NO: 406 - protein, 10 aa, Homo sapiens
QFGNNKTIVF

SEQ ID NO: 407 - protein, 13 aa, Streptococcus sp
QFGREVHAADLLR

SEQ ID NO: 408 - protein, 14 aa, Clostridium tetani
QFIKANSKFIGITE

SEQ ID NO: 409 - protein, 19 aa, Homo sapiens
QFLGQQQPFPPQQPYPQPQ

SEQ ID NO: 410 - protein, 10 aa, Human papillomavirus
QFLRHQNIEF

SEQ ID NO: 411 - protein, 11 aa, Homo sapiens
  Xaa = any amino acid
QFQPFXYFTNT

SEQ ID NO: 412 - protein, 13 aa, Mycobacterium leprae
EEFVVEFALPGIK

SEQ ID NO: 413 - protein, 14 aa, Homo sapiens
QFVIANASSVAKTD

SEQ ID NO: 414 - protein, 17 aa, Homo sapiens
QGALANIAVDKANLEIM

SEQ ID NO: 415 - protein, 13 aa, Homo sapiens
QGARGQPGVMGFP

SEQ ID NO: 416 - protein, 15 aa, Human immunodeficiency virus
QGAYRAIRHIPRRIR

SEQ ID NO: 417 - protein, 15 aa, Simian adenovirus
QGFNNLDNLRDYLDG

SEQ ID NO: 418 - protein, 13 aa, Homo sapiens
QGFQGNPGEPGEP

SEQ ID NO: 419 - protein, 15 aa, Simian adenovirus
QGINNLDILRDYLDG

SEQ ID NO: 420 - protein, 9 aa, Simian adenovirus
QGINNLDNL

SEQ ID NO: 421 - protein, 15 aa, Simian adenovirus
QGINNLDNLRDYLDG

SEQ ID NO: 422 - protein, 24 aa, Plasmodium yoelii
QGPGAPQGPGAPQGPGAPQGPGAP

SEQ ID NO: 423 - protein, 13 aa, Mycobacterium lactis
EEFVVEFALPGIK

SEQ ID NO: 424 - protein, 11 aa, Homo sapiens
QGVHAAHAEIN

SEQ ID NO: 425 - protein, 9 aa, Homo sapiens
QGWKGSPAI

SEQ ID NO: 426 - protein, 10 aa, Human immunodeficiency virus
QGYFPDWQNY

SEQ ID NO: 427 - protein, 11 aa, Clostridium tetani
QIGNDPNRDIL

SEQ ID NO: 428 - protein, 12 aa, Mycobacterium leprae
QIQVYQGEREIA

SEQ ID NO: 429 - protein, 9 aa, Homo sapiens
QIRGRERFE

SEQ ID NO: 430 - protein, 20 aa, Homo sapiens
QITQRKWEAARVAEQDRAYL

SEQ ID NO: 431 - protein, 14 aa, Human immunodeficiency virus
QIVKKLREQFGNNK

SEQ ID NO: 432 - protein, 26 aa, Homo sapiens
QIYPPNANKIREALAQTHSAIAHYWT

SEQ ID NO: 433 - protein, 10 aa, Human immunodeficiency virus
QIYQEPFKNL

SEQ ID NO: 434 - protein, 13 aa, Mycobacterium leprae
EEFVVEFDAPGIK

SEQ ID NO: 435 - protein, 11 aa, Human immunodeficiency virus
QIYQEPFKNLK

SEQ ID NO: 436 - protein, 13 aa, Human immunodeficiency virus
QIYQEPFKNLKTG

SEQ ID NO: 437 - protein, 13 aa, Homo sapiens
QKFTGGIGNKLAA

SEQ ID NO: 438 - protein, 10 aa, Homo sapiens
QKFVACVPGR

SEQ ID NO: 439 - protein, 20 aa, Human immunodeficiency virus
QKGRGSRGQHQAHSLERVCH

SEQ ID NO: 440 - protein, 10 aa, Human immunodeficiency virus
QKLVGKLNWA

SEQ ID NO: 441 - protein, 15 aa, Human immunodeficiency virus
QKLWGKLNWASQIYP

SEQ ID NO: 442 - protein, 16 aa, Human immunodeficiency virus
QKQEPIDKELYPLTSL

SEQ ID NO: 443 - protein, 15 aa, Homo sapiens
QKRAAVDTYCRHNYG

SEQ ID NO: 444 - protein, 15 aa, Homo sapiens
QKRAAYDQYGHAAFE

SEQ ID NO: 445 - protein, 9 aa, Homo sapiens
EADPAGHSY

SEQ ID NO: 446 - protein, 13 aa, Mycobacterium leprae
EEFVVEFDLAGIK

SEQ ID NO: 447 - protein, 16 aa, Homo sapiens
QKRAAYDQYGHAAFEC

SEQ ID NO: 448 - protein, 12 aa, Streptococcus sp
QLAKQAEELAKL

SEQ ID NO: 449 - protein, 9 aa, Homo sapiens
QLAKTCPVQ

SEQ ID NO: 450 - protein, 10 aa, Homo sapiens
QLAKTCPVQL

SEQ ID NO: 451 - protein, 10 aa, Human immunodeficiency virus
QLCKLLRGTK

SEQ ID NO: 452 - protein, 10 aa, Human immunodeficiency virus
QLDCTHLEGK

SEQ ID NO: 453 - protein, 9 aa, Hepatitis B virus
QLFHLCLII

SEQ ID NO: 454 - protein, 9 aa, Hepatitis C virus
QLFTFSPRR

SEQ ID NO: 455 - protein, 10 aa, Human immunodeficiency virus
QLGIPHPAGL

SEQ ID NO: 456 - protein, 11 aa, Homo sapiens
QLIAYLKQATK

SEQ ID NO: 457 - protein, 13 aa, Mycobacterium leprae
EEFVVEFDLPAIK

SEQ ID NO: 458 - protein, 10 aa, Human immunodeficiency virus
QLIKKEKVYL

SEQ ID NO: 459 - protein, 15 aa, Human immunodeficiency virus
QLLFIHFRIGCRHSR

SEQ ID NO: 460 - protein, 10 aa, Hepatitis B virus
QLLWFHISCL

SEQ ID NO: 461 - protein, 13 aa, Sus scrofa
QLNPEMGTDNDSE

SEQ ID NO: 462 - protein, 9 aa, Human immunodeficiency virus
QLQARILAV

SEQ ID NO: 463 - protein, 25 aa, Human immunodeficiency virus
QLQARILAVERYLKDQQLLGIWGCS

SEQ ID NO: 464 - protein, 10 aa, Hepatitis C virus
QLRRHIDLLV

SEQ ID NO: 465 - protein, 20 aa, Epstein -Barr virus
QLSDTPLIPLTIFVGENTGV

SEQ ID NO: 466 - protein, 9 aa, Homo sapiens
QLSLLMWIT

SEQ ID NO: 467 - protein, 9 aa, Mus musculus
QLSPFPFDL

SEQ ID NO: 468 - protein, 13 aa, Mycobacterium leprae
EEFVVEFDLPGAK

SEQ ID NO: 469 - protein, 10 aa, Human immunodeficiency virus
QLTEAVQKIT

SEQ ID NO: 470 - protein, 9 aa, Homo sapiens
QMFCQLAKT

SEQ ID NO: 471 - protein, 9 aa, Homo sapiens
QMLLAIARL

SEQ ID NO: 472 - protein, 11 aa, Human immunodeficiency virus
QMVHQAISPRT

SEQ ID NO: 473 - protein, 10 aa, Homo sapiens
QMVRTAAEVA

SEQ ID NO: 474 - protein, 13 aa, Homo sapiens
QMVRTAAEVAGQL

SEQ ID NO: 475 - protein, 9 aa, Influenza virus
QMVTTTNPL

SEQ ID NO: 476 - protein, 9 aa, Homo sapiens
QMVTTTNPL

SEQ ID NO: 477 - protein, 10 aa, Influenza virus
QMVTTTNPLI

SEQ ID NO: 478 - protein, 9 aa, Homo sapiens
QMWQARLTV

SEQ ID NO: 479 - protein, 13 aa, Mycobacterium leprae
EEFVVEFDLPGIA

SEQ ID NO: 480 - protein, 10 aa, Mus musculus
QNARALDLVA

SEQ ID NO: 481 - protein, 15 aa, Homo sapiens
QNFLLSNAPLGPQFP

SEQ ID NO: 482 - protein, 16 aa, Homo sapiens
QNFTVIFDTGSSNLWV

SEQ ID NO: 483 - protein, 24 aa, Homo sapiens
QNFTVIFDTGSSNLWVPSVYCTSP

SEQ ID NO: 484 - protein, 10 aa, Mus musculus
QNHAALDLVA

SEQ ID NO: 485 - protein, 10 aa, Mus musculus
QNHRAADLVA

SEQ ID NO: 486 - protein, 10 aa, Mus musculus
QNHRALDAVA

SEQ ID NO: 487 - protein, 8 aa, Mus musculus
QNHRALDL

SEQ ID NO: 488 - protein, 10 aa, Mus musculus
QNHRALDLAA

SEQ ID NO: 489 - protein, 10 aa, Mus musculus
QNHRALDLVA

SEQ ID NO: 490 - protein, 13 aa, Mycobacterium leprae
EEFVVEFDLPGIK

SEQ ID NO: 491 - protein, 10 aa, Mus musculus
QNHRALDLVI

SEQ ID NO: 492 - protein, 15 aa, Homo sapiens
QNIFLSNAPLGPQFP

SEQ ID NO: 493 - protein, 15 aa, Homo sapiens
QNIILSNAPLGPQFP

SEQ ID NO: 494 - protein, 15 aa, Homo sapiens
QNILFSNAPLGPQFP

SEQ ID NO: 495 - protein, 15 aa, Homo sapiens
QNILLSNAPLGPQFP

SEQ ID NO: 496 - protein, 15 aa, Homo sapiens
QNILLSNAPQGPQFP

SEQ ID NO: 497 - protein, 15 aa, Homo sapiens
QNILLSNAPVGPQFP

SEQ ID NO: 498 - protein, 15 aa, Homo sapiens
QNILLSNAQLGPQFP

SEQ ID NO: 499 - protein, 15 aa, Homo sapiens
QNILLSNAVLGPQFP

SEQ ID NO: 500 - protein, 15 aa, Homo sapiens
QNILLSNVPLGPQFP

SEQ ID NO: 501 - protein, 9 aa, Homo sapiens
EEFYVDLER

SEQ ID NO: 502 - protein, 15 aa, Homo sapiens [sequence omitted]

SEQ ID NO: 503 - protein, 15 aa, Homo sapiens [sequence omitted]

SEQ ID NO: 504 - protein, 15 aa, Homo sapiens
QNIQLSNAPLGPQFP

SEQ ID NO: 505 - protein, 15 aa, Homo sapiens
QNIVLSNAPLGPQFP

SEQ ID NO: 506 - protein, 15 aa, Homo sapiens
QNVLLSNAPLGPQFP

SEQ ID NO: 507 - protein, 12 aa, Homo sapiens
QPDLRYLFLNGN

SEQ ID NO: 508 - protein, 9 aa, Human papillomavirus
QPFILYAHI

SEQ ID NO: 509 - protein, 9 aa, Homo sapiens
QPFPSQQPY

SEQ ID NO: 510 - protein, 11 aa, Epstein barr virus
QPISHEEQPRY

SEQ ID NO: 511 - protein, 9 aa, Human papillomavirus
QPKKVKRRL

SEQ ID NO: 512 - protein, 9 aa, Influenza virus
EEGAIVGEI

SEQ ID NO: 513 - protein, 11 aa, Epstein barr virus
QPLGTQDQSLY

SEQ ID NO: 514 - protein, 9 aa, Human papillomavirus
QPLTDAKVA

SEQ ID NO: 515 - protein, 11 aa, Epstein barr virus
QPLTSPTTSQL

SEQ ID NO: 516 - protein, 17 aa, Sendai virus
QPMLFKTSIPKLCKAEG

SEQ ID NO: 517 - protein, 8 aa, Homo sapiens
QPQNGQFI

SEQ ID NO: 518 - protein, 15 aa, Lymphocytic choriomeningitis virus
QPQNGQFIHFYREPT

SEQ ID NO: 519 - protein, 9 aa, Staphylococcus erythraeus
QPQRGRENF

SEQ ID NO: 520 - protein, 9 aa, Epstein barr virus
QPRAPIRPI

SEQ ID NO: 521 - protein, 11 aa, Epstein barr virus
QPRAPIRPIPT

SEQ ID NO: 522 - protein, 9 aa, Plasmodium falciparum
QPRPRGDNF

SEQ ID NO: 523 - protein, 9 aa, Mumps virus
EEKLIVVLF

SEQ ID NO: 524 - protein, 15 aa, Human immunodeficiency virus
QQHLLQLTVWGIKQL

SEQ ID NO: 525 - protein, 9 aa, Human papillomavirus type 16
QQLLRREVY

SEQ ID NO: 526 - protein, 9 aa, Homo sapiens
QQLYWSHPR

SEQ ID NO: 527 - protein, 16 aa, Influenza virus
QQRASAGQISVQPAFS

SEQ ID NO: 528 - protein, 14 aa, Homo sapiens
QQRSKILDSIGRFF

SEQ ID NO: 529 - protein, 20 aa, Influenza virus
QQTIIPNIGSRPWVRGLSSR

SEQ ID NO: 530 - protein, 15 aa, Epstein barr virus
QQTNQAGGEAPQPGD

SEQ ID NO: 531 - protein, 16 aa, Homo sapiens
QRARYQWVRCNPDSNS

SEQ ID NO: 532 - protein, 9 aa, Mus musculus
QRGPGRAFV

SEQ ID NO: 533 - protein, 11 aa, Mus musculus
QRGPGRAFVTI

SEQ ID NO: 534 - protein, 13 aa, Streptococcus sp
EELAKQAEELAKL

SEQ ID NO: 535 - protein, 12 aa, Homo sapiens
QRGPGRAFVTIG

SEQ ID NO: 536 - protein, 13 aa, Human immunodeficiency virus
QRGPGRAFVTIGK

SEQ ID NO: 537 - protein, 9 aa, Homo sapiens
QRHGSKYLA

SEQ ID NO: 538 - protein, 10 aa, Human papillomavirus
QRHLDKKQRF

SEQ ID NO: 539 - protein, 16 aa, Homo sapiens
QRKTVKCFNCGKEGHI

SEQ ID NO: 540 - protein, 9 aa, Chlamydia trachomatis
QRLGGGGGK

SEQ ID NO: 541 - protein, 9 aa, Escherichia coli
QRLKEAAEK

SEQ ID NO: 542 - protein, 9 aa, Homo sapiens
QRPGFGYGG

SEQ ID NO: 543 - protein, 11 aa, Homo sapiens
QRVHAAHAEIN

SEQ ID NO: 544 - protein, 9 aa, Homo sapiens
QRYNAMRAA

SEQ ID NO: 545 - protein, 9 aa, Homo sapiens
EELSVLEVF

SEQ ID NO: 546 - protein, 9 aa, Homo sapiens
QRYQKSTEL

SEQ ID NO: 547 - protein, 16 aa, Homo sapiens
QSEAGSHTIQRMYGCD

SEQ ID NO: 548 - protein, 16 aa, Homo sapiens
QSEAGSHTVQRMYGCD

SEQ ID NO: 549 - protein, 13 aa, Homo sapiens
QSEDGSHTIQIMY

SEQ ID NO: 550 - protein, 14 aa, Clostridium tetani
QSIKANSKFIGITE

SEQ ID NO: 551 - protein, 9 aa, Homo sapiens
QSTSRHKKL

SEQ ID NO: 552 - protein, 11 aa, Homo sapiens
QSVHAAHAEIN

SEQ ID NO: 553 - protein, 25 aa, Human immunodeficiency virus
QTGSEELRSLYNTVATLYCVHQRIE

SEQ ID NO: 554 - protein, 9 aa, Plasmodium falciparum
QTNFKSLLR

SEQ ID NO: 555 - protein, 24 aa, Rubella virus
QTPAPKPSRAPPQQPQPPRMQTGR

SEQ ID NO: 556 - protein, 10 aa, Homo sapiens
EADPPTGHSY

SEQ ID NO: 557 - protein, 9 aa, Epstein Barr virus
EENLLDFVR

SEQ ID NO: 558 - protein, 15 aa, Mus musculus
QTQQIRLQAEIFQAR

SEQ ID NO: 559 - protein, 20 aa, Homo sapiens
QTTKHKWEAAHVAEQWRAYL

SEQ ID NO: 560 - protein, 9 aa, Homo sapiens
QVCERIPTI

SEQ ID NO: 561 - protein, 9 aa, Homo sapiens
QVGKYLGLG

SEQ ID NO: 562 - protein, 8 aa, Yersinia sp
QVGNTRTI

SEQ ID NO: 563 - protein, 11 aa, Hepatitis B virus
QVGVGAFGPRL

SEQ ID NO: 564 - protein, 10 aa, Human immunodeficiency virus
QVPLRPHTYK

SEQ ID NO: 565 - protein, 10 aa, Human immunodeficiency virus
QVPLRPMTFK

SEQ ID NO: 566 - protein, 10 aa, Human immunodeficiency virus
QVPLRPMTSK

SEQ ID NO: 567 - protein, 10 aa, Human immunodeficiency virus
QVPLRPMTYK

SEQ ID NO: 568 - protein, 10 aa, Epstein barr virus
EENLLDFVRF

SEQ ID NO: 569 - protein, 10 aa, Human immunodeficiency virus
QVRDQAEHLK

SEQ ID NO: 570 - protein, 13 aa, Homo sapiens
QVVALKPAIAAAA

SEQ ID NO: 571 - protein, 11 aa, Homo sapiens
QVVHAAHAEIN

SEQ ID NO: 572 - protein, 15 aa, Human immunodeficiency virus
QWQNYTPGPGVRYPL

SEQ ID NO: 573 - protein, 14 aa, Homo sapiens
QYAKANSKFIGITE

SEQ ID NO: 574 - protein, 9 aa, Homo sapiens
QYDAAVYKL

SEQ ID NO: 575 - protein, 9 aa, Homo sapiens
QYDDAGYKL

SEQ ID NO: 576 - protein, 9 aa, Homo sapiens
QYDDAVAKL

SEQ ID NO: 577 - protein, 9 aa, Homo sapiens
QYDDAVEKL

SEQ ID NO: 578 - protein, 9 aa, Homo sapiens
QYDDAVYDL

SEQ ID NO: 579 - protein, 20 aa, Epstein Barr virus
EENLLDFVRFMGVMSSCNNP

SEQ ID NO: 580 - protein, 9 aa, Homo sapiens
QYDDAVYEL

SEQ ID NO: 581 - protein, 9 aa, Homo sapiens
QYDDAVYFL

SEQ ID NO: 582 - protein, 9 aa, Homo sapiens
QYDDAVYHL

SEQ ID NO: 583 - protein, 9 aa, Homo sapiens
QYDDAVYKF

SEQ ID NO: 584 - protein, 9 aa, Homo sapiens
QYDDAVYKL

SEQ ID NO: 585 - protein, 9 aa, Homo sapiens
QYDDAVYRL

SEQ ID NO: 586 - protein, 9 aa, Homo sapiens
QYDDAVYSL

SEQ ID NO: 587 - protein, 9 aa, Homo sapiens
QYDDRVYKL

SEQ ID NO: 588 - protein, 9 aa, Homo sapiens
QYDEAVAQF

SEQ ID NO: 589 - protein, 11 aa, Plasmodium falciparum
QYDLFIYNKQL

SEQ ID NO: 590 - protein, 16 aa, Homo sapiens
EENLRFDSDVGEFRAV

SEQ ID NO: 591 - protein, 9 aa, Homo sapiens
QYDQIPVEI

SEQ ID NO: 592 - protein, 14 aa, Clostridium tetani
QYIAANSKFIGITE

SEQ ID NO: 593 - protein, 14 aa, Clostridium tetani
QYIIANSKFIGITE

SEQ ID NO: 594 - protein, 14 aa, Clostridium tetani
QYIKAASKFIGITE

SEQ ID NO: 595 - protein, 15 aa, Clostridium tetani
QYIKAASKFIGITEL

SEQ ID NO: 596 - protein, 14 aa, Clostridium tetani
QYIKALSKFIGITE

SEQ ID NO: 597 - protein, 14 aa, Clostridium tetani
QYIKANAKFIGITE

SEQ ID NO: 598 - protein, 14 aa, Clostridium tetani
QYIKANQKFIGITE

SEQ ID NO: 599 - protein, 14 aa, Clostridium tetani
QYIKANSAFIGITE

SEQ ID NO: 600 - protein, 14 aa, Clostridium tetani
QYIKANSEFIGITE

SEQ ID NO: 601 - protein, 16 aa, Homo sapiens
EENVEHDAEENVEHDA

SEQ ID NO: 602 - protein, 13 aa, Clostridium tetani
QYIKANSFIGITE

SEQ ID NO: 603 - protein, 14 aa, Clostridium tetani
QYIKANSKAIGITE

SEQ ID NO: 604 - protein, 14 aa, Clostridium tetani
QYIKANSKEIGITE

SEQ ID NO: 605 - protein, 14 aa, Clostridium tetani
QYIKANSKFAGITE

SEQ ID NO: 606 - protein, 14 aa, Clostridium tetani
QYIKANSKFIAITE

SEQ ID NO: 607 - protein, 14 aa, Clostridium tetani
QYIKANSKFIGATE

SEQ ID NO: 608 - protein, 12 aa, Clostridium tetani
QYIKANSKFIGI

SEQ ID NO: 609 - protein, 14 aa, Clostridium tetani
QYIKANSKFIGIAE

SEQ ID NO: 610 - protein, 14 aa, Clostridium tetani
QYIKANSKFIGIFE

SEQ ID NO: 611 - protein, 14 aa, Clostridium tetani
QYIKANSKFIGIKE

SEQ ID NO: 612 - protein, 11 aa, Mus musculus
EEQTQQIRLQA

SEQ ID NO: 613 - protein, 14 aa, Clostridium tetani
QYIKANSKFIGISE

SEQ ID NO: 614 - protein, 13 aa, Clostridium tetani
QYIKANSKFIGIT

SEQ ID NO: 615 - protein, 14 aa, Clostridium tetani
QYIKANSKFIGITA

SEQ ID NO: 616 - protein, 14 aa, Clostridium tetani
QYIKANSKFIGITD

SEQ ID NO: 617 - protein, 14 aa, Clostridium tetani
QYIKANSKFIGITE

SEQ ID NO: 618 - protein, 15 aa, Clostridium tetani
QYIKANSKFIGITEL

SEQ ID NO: 619 - protein, 17 aa, Clostridium tetani
QYIKANSKFIGITELKK

SEQ ID NO: 620 - protein, 19 aa, Clostridium tetani
QYIKANSKFIGITELKKLE

SEQ ID NO: 621 - protein, 14 aa, Clostridium tetani
QYIKANSKFIGITK

SEQ ID NO: 622 - protein, 14 aa, Clostridium tetani
QYIKANSKFIGITK

SEQ ID NO: 623 - protein, 13 aa, Mus musculus
EEQTQQIRLQAEI

SEQ ID NO: 624 - protein, 14 aa, Clostridium tetani
QYIKANSKFIGITV

SEQ ID NO: 625 - protein, 14 aa, Clostridium tetani
QYIKANSKFIGKTE

SEQ ID NO: 626 - protein, 14 aa, Clostridium tetani
QYIKANSKFIGLTE

SEQ ID NO: 627 - protein, 14 aa, Clostridium tetani
QYIKANSKFIGQTE

SEQ ID NO: 628 - protein, 14 aa, Clostridium tetani
QYIKANSKFIQITE

SEQ ID NO: 629 - protein, 14 aa, Clostridium tetani
QYIKANSKFIQITE

SEQ ID NO: 630 - protein, 14 aa, Clostridium tetani
QYIKANSKFIYITE

SEQ ID NO: 631 - protein, 14 aa, Clostridium tetani
QYIKANSKFKGITE

SEQ ID NO: 632 - protein, 14 aa, Clostridium tetani
QYIKANSKFQGITE

SEQ ID NO: 633 - protein, 14 aa, Clostridium tetani
QYIKANSKFVGITE

SEQ ID NO: 634 - protein, 14 aa, Mus musculus
EEQTQQIRLQAEIF

SEQ ID NO: 635 - protein, 14 aa, Clostridium tetani
QYIKANSKSIGITE

SEQ ID NO: 636 - protein, 14 aa, Clostridium tetani
QYIKANSKYIGITE

SEQ ID NO: 637 - protein, 14 aa, Clostridium tetani
QYIKANSRFIGITE

SEQ ID NO: 638 - protein, 14 aa, Clostridium tetani
QYIKANSSFIGITE

SEQ ID NO: 639 - protein, 14 aa, Clostridium tetani
QYIKANYKFIGITE

SEQ ID NO: 640 - protein, 14 aa, Clostridium tetani
QYIKAQSKFIGITE

SEQ ID NO: 641 - protein, 14 aa, Clostridium tetani
QYIKFNSKFIGITE

SEQ ID NO: 642 - protein, 14 aa, Clostridium tetani
QYIKKNSKFIGITE

SEQ ID NO: 643 - protein, 14 aa, Clostridium tetani
QYIKSNSKFIGITE

SEQ ID NO: 644 - protein, 14 aa, Clostridium tetani
QYIRANSKFIGITE

SEQ ID NO: 645 - protein, 15 aa, Mus musculus
EEQTQQIRLQAEIFQ

SEQ ID NO: 646 - protein, 14 aa, Clostridium tetani
QYISANSKFIGITE

SEQ ID NO: 647 - protein, 14 aa, Clostridium tetani
QYKKANSKFIGITE

SEQ ID NO: 648 - protein, 9 aa, Homo sapiens
QYLAGLSTL

SEQ ID NO: 649 - protein, 9 aa, Homo sapiens
QYLALAALI

SEQ ID NO: 650 - protein, 9 aa, Plasmodium falciparum
QYLKKIKNS

SEQ ID NO: 651 - protein, 20 aa, Plasmodium falciparum
QYLKKIKNSISTEWSPCSVT

SEQ ID NO: 652 - protein, 10 aa, Plasmodium falciparum
QYLKKIKNSL

SEQ ID NO: 653 - protein, 13 aa, Homo sapiens
QYMRADQAAGGLR

SEQ ID NO: 654 - protein, 10 aa, Human papillomavirus
QYNKPLCDDL

SEQ ID NO: 655 - protein, 9 aa, Homo sapiens
QYNVLPQGW

SEQ ID NO: 656 - protein, 17 aa, Mus musculus
EEQTQQIRLQAEIFQAR

SEQ ID NO: 657 - protein, 14 aa, Clostridium tetani
QYQKANSKFIGITE

SEQ ID NO: 658 - protein, 11 aa, Homo sapiens
QYRRALDLVAA

SEQ ID NO: 659 - protein, 11 aa, Homo sapiens
QYVHAAHAEIN

SEQ ID NO: 660 - protein, 14 aa, Clostridium tetani
QYVKANSKFIGITE

SEQ ID NO: 661 - protein, 10 aa, Homo sapiens
QYVKQNTLKL

SEQ ID NO: 662 - protein, 9 aa, Homo sapiens
EADPTAHSY

SEQ ID NO: 663 - protein, 15 aa, Human immunodeficiency virus
EESQNQQEKNEQELL

SEQ ID NO: 664 - protein, 8 aa, Influenza virus
EESTGNLI

SEQ ID NO: 665 - protein, 15 aa, Homo sapiens
EEVDMTPADALDDFD

SEQ ID NO: 666 - protein, 11 aa, Human immunodeficiency virus
EEVGFPVRPQV

SEQ ID NO: 667 - protein, 15 aa, Human immunodeficiency virus
EFFYCNTTQLFNNTW

SEQ ID NO: 668 - protein, 11 aa, Homo sapiens
EFISEAIIHVL

SEQ ID NO: 669 - protein, 9 aa, Homo sapiens
EFQAAISRK

SEQ ID NO: 670 - protein, 8 aa, Homo sapiens
EFVNTPPL

SEQ ID NO: 671 - protein, 12 aa, Homo sapiens
EFWEFDLPGIKA

SEQ ID NO: 672 - protein, 8 aa, Influenza virus
EGAIVGEI

SEQ ID NO: 673 - protein, 9 aa, Homo sapiens
EADPTGASY

SEQ ID NO: 674 - protein, 15 aa, Homo sapiens
EGFSYTDANKNKGIV

SEQ ID NO: 675 - protein, 8 aa, Influenza A virus
EGGWTGMI

SEQ ID NO: 676 - protein, 12 aa, Influenza virus
EGILGFVFTLTV

SEQ ID NO: 677 - protein, 17 aa, Plasmodium malariae
EGKIAKMEKASSVFNVV

SEQ ID NO: 678 - protein, 13 aa, Homo sapiens
EGMRFDKGYISGY

SEQ ID NO: 679 - protein, 20 aa, Homo sapiens
EGQLVSIHSPEEQDFLTKHA

SEQ ID NO: 680 - protein, 9 aa, Homo sapiens
EGQRPGFGY

SEQ ID NO: 681 - protein, 9 aa, Homo sapiens
EHAGVISVL

SEQ ID NO: 682 - protein, 23 aa, Homo sapiens
EHHIFLGATNYIYVLNEEDLQKV

SEQ ID NO: 683 - protein, 17 aa, Homo sapiens
EHPSLQSPITVEWRLLH

SEQ ID NO: 684 - protein, 9 aa, Homo sapiens
EADPTGHAY

SEQ ID NO: 685 - protein, 15 aa, Mycobacterium tuberculosis
EHRVKRGLTVAVAGA

SEQ ID NO: 686 - protein, 13 aa, Homo sapiens
EIAYDICRRNLDI

SEQ ID NO: 687 - protein, 17 aa, Homo sapiens
EIAYDICRRNLDIERPT

SEQ ID NO: 688 - protein, 17 aa, Human immunodeficiency virus type 1
EICTEMEKEGKISKIGP

SEQ ID NO: 689 - protein, 9 aa, Human papillomavirus type 16
EIDGPAGQA

SEQ ID NO: 690 - protein, 16 aa, Human immunodeficiency virus
EIDNYTNTIYTLLEEC

SEQ ID NO: 691 - protein, 10 aa, Homo sapiens
EIKANSKFIG

SEQ ID NO: 692 - protein, 9 aa, Human immunodeficiency virus
EIKDTKEAL

SEQ ID NO: 693 - protein, 15 aa, Human immunodeficiency virus
EIKDTKEALDKIEEE

SEQ ID NO: 694 - protein, 16 aa, Human immunodeficiency virus
EIKILNIFGVIKGFVE

SEQ ID NO: 695 - protein, 25 aa, Human immunodeficiency virus
  MUTAGEN (25): Xaa = Lys branched with a Palm-NH2 moiety
EWRFDSRLAFHHVAREHPEYFNKNX

SEQ ID NO: 696 - protein, 10 aa, Homo sapiens
  MOD_RES (1): PYRROLIDONE CARBOXYLIC ACID
XLAGIGILTV

SEQ ID NO: 697 - protein, 10 aa, Homo sapiens
  MUTAGEN (1): Xaa = glutamic acid acetylated on amine functional group
XLAGIGILTV

(19) United States
(12) Patent Application Publication
(10) Pub. No.: US 2003/0175285 A1
(43) Pub. Date: Sep. 18, 2003

(54) Molecule of pharmaceutical interest comprising at its n-terminal a glutamic acid or a glutamine in the form of a physiologically acceptable strong acid

(76) Inventors: Christine Klinguer-Hamour, Groisy (FR); Corvaia Nathalie, Genevois (FR); Beck Alain, Saleve (FR); Goetsch Liliane, Ayze (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/239,313

(22) PCT Filed: Mar. 22, 2001
(86) PCT No.: PCT/FR01/00872

(30) Foreign Application Priority Data
Mar. 23, 2000 (FR) 00/03711

(51) Int. Cl. A61K 39/00; C07K 14/74

(52) U.S. Cl. 424/185.1; 514/12; 530/350; 530/359

(57) ABSTRACT

The invention concerns a molecule of pharmaceutical interest, preferably a major histocompatibility complex (MHC) ligand, comprising a glutamic acid or a glutamine at its N-terminal, in the form of a physiologically acceptable addition salt, and a vaccine comprising such a ligand.

The subject of the present invention is a molecule of pharmaceutical interest, preferably a ligand for the Major Histocompatibility Complex (MHC), containing a glutamic acid or a glutamine at its N-terminal end, which exists in the form of a physiologically acceptable addition salt with a strong acid, and a vaccine comprising such a ligand.

Vaccination is an effective means for preventing or reducing viral or bacterial infections. The vaccine antigens when administered alone to the host are often not sufficiently immunogenic to induce an immune response, and should therefore be combined with an adjuvant or coupled to a carrier protein in order to elicit (or increase) their immunogenicity. Under these conditions, only a humoral type immune response may be induced. However, in the context of an antiviral therapy, the generation of cytotoxic T lymphocytes (CTL) capable of recognizing and destroying the virus is of primary importance (Bachmann et al., Eur. J. Immunol., 1994, 24, 2228-2236; Borrow P., J. Virol. Hepat., 1997, 4, 16-24), as demonstrated by numerous studies showing in vivo the protective role of the responses directed against the viral epitopes (Arvin A. M., J. Inf. Dis., 1992, 166, pp. 35-41; Koszinowski et al., Immunol. Lett., 1987, 16, 185-192).

The importance of the CTL and helper T responses has also been indeed described for vaccines against parasites such as Plasmodium falciparum, the agent responsible for Malaria (Le et al., Vaccine, 1998, 16, 305-312).

The vital role of the CTL responses has also been greatly documented in antitumor responses, in particular those directed against melanoma cells (review in Rivoltini et al., Crit. Rev. Immunol., 1998, 18, 55-63). The CTL epitope(s) (peptide sequences interacting with the class I molecules and presented to the CD8+ T lymphocytes) have been defined for several antigens. However, the difficulty lies in the generation of CTL in vivo, due to the low immunogenicity of these peptides (Melief, Adv. Cancer Res., 1992, 58, 143-175; Nandaz and Sercaz, Cell, 1995, 82, 13-17).

Numerous ligands for the MHC (class I and II) and in particular for the CTL epitope peptides have been identified (HG Rammensee et al., Immunogenetics, 1999, 50, 213) and some of their sequences are accessible on the Internet in public databases. There may be mentioned in particular the bases SYFPEITHI (http://www.uni-tuebingen.de/uni/kxi/) and MHCPEP (http://wehih.wehi.edu.au/mhcpep/). Likewise, supertypes of the principal HLAs have been described (Sette et al., Immunogenetics, 1999, 50, 201-212).

The importance of these MHC ligands is confirmed by the increasing number of clinical studies in humans of these compounds as candidate vaccines against various pathologies and in particular as anti-melanoma vaccines (epitopes m27-25 MART 1, g209-217, g280-288, gp100, MAGE 3), as anti-HIV vaccine (Klinguer et al., Vaccine, 2000, 18, 259-267) or as anti-HBV vaccines of the anti-HBV lipopeptide type (Livingston et al., J. Immunol., 1999, 162, 3088-3095).

However, the difficulty of these studies lies in the fact that the peptides used are difficult to preserve before their administration to the patients, which can lead to a reduction in their vaccine power, and to a more rapid degradation in vivo.

To stabilize a peptide intended for pharmaceutical use which has a glutamic acid or a glutamine at the N-terminus in the form of a salt compatible with administration to humans, the strategy normally used by persons skilled in the art is to synthesize the pyroglutamic derivative of this peptide, as the two examples below of Buserelin and Gonadorelin (LH-RH analogs, European Pharmacopoeia, 1999) illustrate:

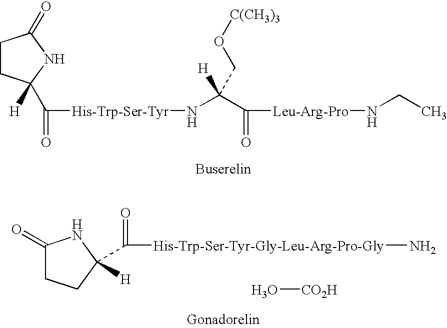

This moreover makes it possible to increase the half-life of the peptide by limiting its proteolytic degradation by N-aminopeptidases.

However, when this method is used to stabilize an MHC ligand such as the ELA decapeptide (CTL epitope of sequence ELAGIGILTV and of formula C₄₅H₈₀N₁₀O₁₄₌985 Da), the PyrELA derivative obtained (of sequence PyrELAGIGILTV and of formula C₄₅H₇₈N₁₀O₁₃₌967 Da) no longer exhibits the desired vaccine activity and is in particular practically inactive from the point of view of a CTL response. This structural modification is nevertheless minor: it involves the cyclization of the N-terminal α-amino functional group of the glutamic acid with its own γ-carboxylic functional group and loss of a molecule of water. Indeed, the peptides having an amino acid of the glutamic acid (Glu, E) or glutamine (Gln, Q) type at their N-terminal end cyclize with the free γ-carboxylic acid functional group to form a pyroglutamate according to the reaction defined below:

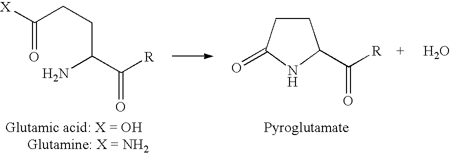

The absence of a vaccine activity for these peptides is all the more surprising since the reduction in mass between the ELA decapeptide and the PyrELA derivative obtained is only 18 Daltons, while the remainder of the structure remains unchanged:

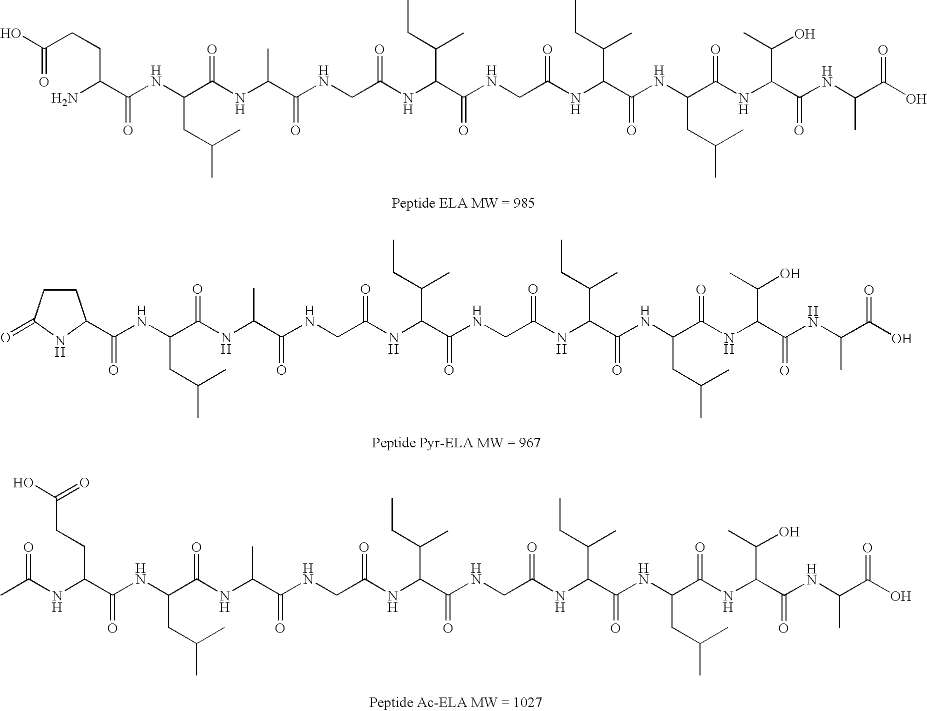

It has also been observed that the synthesis of another derivative of the ELA peptide acetylated on the amine functional group of the glutamic acid so as to prevent cyclization to pyroglutamate (AcELA peptide, of sequence AcELAGIGILTV and of formula C₄₇H₈₂N₁₀O₁₅₌1027 Da, see above) makes it possible to solve the problem of stability but causes the AcELA derivative thus obtained to lose the entire vaccine activity, and in particular the CTL cell generating activities.

This acetylation reaction is nevertheless a minor modification of the structure of the peptide conventionally used by persons skilled in the art to improve the stability of a peptide (Brinckerhoff et al., Int . J. Cancer, 1999, 83, 326): it involves the replacement of one of the protons of the N-terminal NH₂ functional group by an acetyl group CH₃CO with a small increase in mass (42 Da over 985 Da), the remainder of the structure remaining unchanged.

Likewise, Elliott et al. (Vaccine, 1999, 17, 2009-2019) have described problems of stability of CTL epitopes containing methionines (oxidation to a sulfoxide) or glutamic acids at the N-terminal position (peptide EEGAIVGEI, derived from the influenza protein NSP-1 of the influenza virus (amino acids 152-160) and corresponding to a restricted H-2Kk mouse CTL epitope). It was observed that this peptide cyclizes spontaneously to pyroglutamate (30% in 2 months) when it is formulated with an adjuvant solution of the Montanide ISA 720 type. The authors raise the problem that this degradation poses with respect to the desired vaccine activity, without providing a solution thereto.

In addition, practically all the peptides obtained by chemical synthesis are purified by reversed-phase HPLC with the aid of eluents containing trifluoroacetic acid (TFA) before being freeze-dried. The purified peptides obtained are positively charged and exist in the form of a trifluoroacetate salt (RNH₃⁺,CF₃CO₂⁻). The quantity of trifluoroacetate and of residual trifluoroacetic acid is in general proportional to the number of basic amino acids (Lysine, Arginine and Histidine) contained in the sequence as well as the amine functional group of the N-terminal amino acid. Peptides in trifluoroacetate form are commonly used for preclinical experiments in vitro and in vivo in animals. For a pharmaceutical use in humans, this salt form is however not accepted in particular during the final stages of purifications because trifluoroacetic acid is part of a class of solvents (class IV) whose toxicology is not perfectly documented (Leblanc et al., STP Pharma, 1999, 9, 334-341). Thus, none of the peptides which have obtained a marketing authorization (Somatostatin, Tetracoside, Desmopressin, Calcitonin, Buserelin, Gonadorelin, and the like) were in the trifluoroacetate form, as may be observed in the European Pharmacopoeia monographs (Ph. Eur. 1999), but rather in the acetate form. The quantity of residual trifluoroacetic acid tolerated in these peptides is in fact extremely limited.

Moreover, a recent study (Cornish et al., Am. J. Physiol. Endocrinol. Metab., 1999, 277, E779-E783) has shown that several synthetic peptides (Amylin, Calcitonin) in trifluoroacetate form are toxic for cells in culture (osteoblasts and chondrocytes).

A solution for solving these various problems of toxicity of trifluoroacetic acid has been proposed by Marchand et al. (Int. J. Cancer, 1999, 80, 219-230), who report results of a clinical study demonstrating a tumor regression in patients suffering from a melanoma. The active ingredient used is the nonapeptide MAGE-3 having the sequence EVDPIGHLY (SEQ ID No. 273), which possesses a glutamic acid at the N-terminal. The peptide was used in patients in the acetate form which is the form used in practically all the peptides administered to humans.

However, acetic acid is a weak acid, which confers increased instability on the peptide. This forces investigators to store the peptide at −80° C. (liquid nitrogen) in freeze-dried form and to resolubilize it immediately before the injection, which involves a highly constraining cold chain.

The present invention proposes to solve these problems of structural instability, of preservation over time, of toxicity and of biological activity.

Indeed, it has been observed, surprisingly, that the molecules of pharmaceutical interest, in particular the MHC ligands, possessing a glutamic acid or a glutamine at their N-terminal end can be stabilized in the form of an addition salt with a strong acid, and that this makes it possible both to maintain the biological activity and to obtain easy preservation of the peptide or analog in a stable form, which allows its therapeutic use in humans.

The expression “molecule of pharmaceutical interest” is understood to mean in particular the MHC ligands, the natural or synthetic molecules having an epitope for the generation of antibodies, the molecules derived from receptor ligands, and exhibiting an agonist or antagonist activity with respect to these receptors, or possessing an antibiotic, antifungal or antiviral activity. The molecules of therapeutic interest according to the invention are all characterized in that they possess a glutamic acid or a glutamine at their N-terminal end. The preferred molecules of pharmaceutical interest according to the present invention are the MHC ligands.

The subject of the present invention is thus in particular an MHC ligand containing at its N-terminal end a glutamic acid or a glutamine, characterized in that it exists in the form of a physiologically acceptable addition salt with a strong acid.

The physiologically acceptable addition salt with a strong acid may be chosen in particular from the addition salts with strong inorganic or organic acids.

It is preferably chosen from the methanesulfonate (or mesilate), hydrochloride, hydrobromide, sulfate, nitrate and phosphate and more preferably from the hydrochloride, sulfate, nitrate and methanesulfonate.

These addition salts with a strong acid are physiologically acceptable for a therapeutic use in humans. For example, Protamine (obtained by extraction from sperm or from soft roe of fish and which requires a strong acid salt in order to be solubilized) is registered in the hydrochloride form, on the one hand, and in the sulfate form, on the other (Ph. Eur., 1999).

The MHC ligands for the purposes of the present invention are in particular the MHC class I and II ligands. MHC is an important group of proteins involved in the presentation of antigens to the T lymphocytes. The MHC class I molecules are integral membrane proteins which are found on all nucleated cells and the platelets. The MHC class II molecules are expressed on the B cells, the macrophages, the monocytes, the antigen-presenting cells and certain T cells. The B cells are lymphocytes which, in a mature form, present at their surface immunoglobulins acting as “receptor for the antigen”. The T cells are lymphocytes which express their receptor for the antigen (TcR) and are differentiated into 2 subpopulations: T helper cells (Th or T helper) and cytotoxic T cells (CTL). The Th cells help the B cells to divide, to differentiate and to produce antibodies. The majority of the Th cells are CD4+ (specific surface marker) and recognize the antigen presented at the surface of the antigen-presenting cells, in combination with the MHC class II molecules. The cytotoxic T cells are capable of destroying the target cells infected by viruses or allogenic cells. The majority are CD8+ and recognize the antigen associated with the MHC class I molecules at the surface of the target cell. The recognition of the antigen occurs by formation of a complex comprising in particular the MHC molecule presenting an MHC ligand, and the T cell receptor (TCR).

The molecules of pharmaceutical interest, in particular the MHC ligands according to the present invention, may be chosen from natural or synthetic molecules, and, inter alia, from proteins, peptides, multi-epitope polypeptide constructs, or peptide analogs of the pseudopeptide, retro-inverso or peptoid type, peptido-mimetics, and lipopeptides. These molecules may also partly consist of a peptide chain, with the replacement of certain amino acids by amino acid analogs, or a chain having branches. These molecules may also exhibit various modifications which are observed on the natural proteins or peptides (for example O- or N-glycosylation).

In a preferred embodiment of the invention, the MHC ligands according to the present invention are chosen from the CTL epitopes, that is to say those which allow the generation of cytotoxic T lymphocytes, and in particular from those which exist in the form of an octapeptide, a nonapeptide or a decapeptide.

The MHC ligand may also be chosen from the ligands described in the databases SYFPEITHI or MHCPEP, cited above, and which contain, at their N-terminal end, a glutamic acid or a glutamine.

This ligand may be chosen in particular from the MHC ligands (ligands for the MHC class I or II molecules) included in the group consisting of the peptides corresponding to the sequences SEQ ID No. 1 to SEQ ID No. 694.

In an embodiment of the invention which is even more particularly preferred, it is chosen from the following peptides:

| | | | |
|---|---|---|---|
| | | | SEQ ID |
| Names | Sequences | HLA | No. |

| | | | | | |
|---|---|---|---|---|---|
| ELA MART-1 | 26-35 | A27L | ELAGIGILTV | A2 | 81 |
| ELA MART-1 | 26-35 | | EAAGTGILTV | A2 | 112 |
| MAGE-1 | 161-169 | | EADPTGHSY | A1 | 2 |
| MAGE-3 | 168-176 | | EVDPIGHLY | A1 | 273 |
| HER-2/neu | 950-958 | | ELVSEFSPRM | A2 | 110 |
| HCV-1 env E | 66-75 | | QLRRHTDLLV | A2 | 464 |
| NY-ESO-1 | 155-163 | | QLSLLMWIT | A2 | 466 |
| HIV nef | 73-82 | | QVPLRPMTYK | A3 | 567 |
| Influenza NP | 380-388 | | ELRSRYWAI | B8 | 166 |
| HIV gag p24 | 262-270 | EIYKRWIIL | B8 | 10 | |
| HIV gag p17 | 93-101 | EIKDTKEAL | B8 | 692 | |
| Influenza NP | 339-347 | | EDLRVLSFI | B*3701 | 257 |
| EBNA 6 | 130-139 | | EENLLDFVRF | B*4403 | 568 |

The ligands according to the invention may also be chosen from the multi-epitope polypeptide constructs having an amino acid of the glutamic acid (Glu, E) or glutamine (Gln, Q) type at the N-terminal end such as the following peptide (SEQ ID No. 695):

NEF 117 EWRFDSRLAFHHVAREHPEYFNKNK(Palm)NH₂ (anti-HIV lipopeptide in clinical phase I: Klinguer, et al., Vaccine, 1999, 18, 259-267).

The peptide analogs may be chosen from those described in application FR276307 which contain, at their N-terminal end, a glutamic acid or a glutamine.

More preferably, the invention relates to the MHC ligand having the sequence ELAGIGILTV, in sulfate form or, even more preferably, in hydrochloride form.

The invention also relates to a pharmaceutical composition comprising at least one molecule of pharmaceutical interest according to the invention.

This pharmaceutical composition may in particular be intended for the treatment of various immunopathologies: immunodeficiency, autoimmune diseases, hypersensitivities, allergies or for avoiding graft rejections. Depending on the molecule used, a composition according to the invention may also be used for an antibiotic, antiviral or antifungal purpose, or may be intended for the treatment of diseases linked to hormonal disruptions, or to diseases of the central nervous system.

The compositions according to the invention may also be used in the veterinary field. Indeed, the same problems of structural instability, of preservation over time, of toxicity and of activity which are posed for the preparation of veterinary preparations comprising a peptide or a molecule possessing a glutamic acid or a glutamine at their N-terminal end may be solved using addition salts with strong acids to stabilize said peptides or molecules.

Among the pharmaceutical compositions according to the invention, a preferred composition consists of a vaccine, characterized in that it comprises at least one MHC ligand according to the invention, existing in the form of a physiologically acceptable addition salt with a strong acid, as defined above.

This vaccine may comprise, in addition, at least one adjuvant, in particular chosen from the salts of Aluminum (Alum) or of Calcium, the enterobacterial OmpA proteins, the tetanus toxoid (TT), the diphtheria toxoid (DT), CRM197 (cross-reactivity material), PLGA, ISCOM, Montanide ISA 720, aliphatic quaternary ammoniums, MPL-A, Quil-A, CpGs, Leif, the cholera toxin (CT), LT (LT for “heat labile enterotoxin”) or the detoxified versions of CT or LT.

In a preferred form of the invention, the vaccine comprises, in addition, a carrier compound mixed with or coupled to said ligand.

Preferably, said carrier compound is chosen from the peptide group comprising toxoids, in particular the diphtheria toxoid (DT) or the tetanus toxoid (TT), proteins derived from streptococcus (such as the human seralbumin binding protein, called “BB”, described in WO96/14415), membrane proteins OmpA (for “outer membrane protein type A” and the outer membrane protein complexes (OMPC), outer membrane vesicles (OMV) or heat-shock proteins (HSP).

Advantageously, said carrier compound is covalently coupled with the ligand. The expression “coupling” is intended to designate both a coupling achieved by a chemical route between the two compounds, and a biological coupling, by genetic recombination, as defined below.

Thus, according to the invention, it is possible to introduce one or more linking elements, in particular amino acids, in order to facilitate the coupling reactions between the carrier compound and the antigen or hapten, in particular. when they are of a peptide nature, it being possible for the covalent coupling of the antigen or hapten to be carried out at the N- or C-terminal end of the carrier compound.

The bifunctional reagents allowing this coupling are determined according to the end of the carrier compound chosen and the nature of the antigen or hapten to be coupled. These coupling techniques are well known to persons skilled in the art.

The conjugates derived from a coupling of peptides may also be prepared by genetic recombination. The hybrid peptide (conjugate) may indeed be produced by recombinant DNA techniques by insertion into or addition to the DNA sequence encoding the carrier compound, of a sequence encoding the antigenic, immunogenic or hapten peptide or peptides. These techniques for preparing a hybrid peptide by genetic recombination are well known to persons skilled in the art (cf. for example Makrides, 1996, Microbiologicals Reviews, 60, 512-538).

Preferably, said carrier compound is a protein derived from streptococcus or a membrane protein OmpA from an enterobacterium, in particular from Klebsiella pneumoniae, or one of its fragments.

The ligand according to the invention, optionally combined with a carrier compound, may be incorporated into vectors chosen from liposomes, virosomes, nanospheres, microspheres, microcapsules or biovectors. Persons skilled in the art know how to choose the appropriate vector according to the desired aim (protection of the ligand optionally combined with a carrier compound or an adjuvant for the degradation, targeting of cells of interest, search for penetration of the material contained in the vector inside target cells, and the like).

One embodiment of the invention comprises in particular an anti-melanoma vaccine, characterized in that it comprises at least one peptide ELAGIGILTV (SEQ ID No. 81) in hydrochloride or sulfate form.

The subject of another embodiment is an anti-melanoma vaccine, characterized in that it comprises at least one peptide ELAGIGILTV (SEQ ID No. 81) in hydrochloride or sulfate form and, in addition, an enterobacterial OmpA protein.

It is also possible to develop vaccines according to the invention for use in the veterinary field, it being possible for the identical problems of structural instability, preservation over time, toxicity and activity to be solved in the same manner.

The subject of the invention is also a method for the in vitro diagnosis of pathologies associated with the presence, in a patient's body, of MHC ligands which can interact with MHC molecules, and which may be directly or indirectly involved in the process of development of these pathologies in humans or animals, characterized in that it comprises the steps of:

bringing a biological sample obtained from a patient, in particular blood or any biological sample which may contain lymphocytes, into contact with an MHC ligand according to the invention, under conditions allowing the formation of a binary complex between said MHC ligand and the MHC molecules present in said sample, and the reaction between said binary complex and the T cell receptors which may be present in said biological sample,

detecting in vitro the ternary complex MHC—MHC ligand—T receptor, which may be formed i the preceding step.

The diagnostic methods according to the invention are advantageously carried out in the following manner:

incubation of said biological sample with MHC ligands according to the invention, said MHC ligands being attached to a solid support, in particular inside wells of microtiter plates of the type normally used for carrying out detection or assay techniques well known under the name ELISA (Enzyme Linked Immuno Sorbent Assay),

incubation of the components attached to the solid support, after an optional rinsing step, with a medium containing antibodies, in particular anti-ternary complex antibodies according to the invention, labeled (in particular radioactively, enzymatically or by fluorescence), or which may be recognized in turn by a labeled reagent,

detection of the labeled antibodies which have remained respectively attached to the ternary complexes during the preceding incubation step.

Rinsing steps are advantageously carried out between the different steps of this method. Persons skilled in the art know how to define the various incubation conditions, as well as the methods for detecting MHC—MHC ligand—T receptor complexes, the use of antibodies being only one method among others.

The subject of the invention is also the packs or kits for carrying out in vitro diagnostic methods as described above, comprising:

an MHC ligand according to the invention;

optionally reagents to allow the formation of an immunological reaction between said ligand, the MHC molecules and the T cell receptors which may be present in the biological sample;

optionally reagents which make it possible to detect the ternary complex according to the invention, which was produced at the end of the immunological reaction, said reagents optionally containing a marker or being capable of being recognized in turn by a labeled reagent, more particularly in the case where the peptide analog is not labeled.

In particular, the use of the peptides ELAGIGILTV (SEQ ID No. 81), EAAGIGILTV (SEQ ID No. 112), EADPTGHSY (SEQ ID No. 2), or EVDPIGHLY (SEQ ID No. 273) is preferred in a method for the diagnosis of a melanoma. The peptides QVPLRPMTYK (SEQ ID No. 567), EIYKRWIIL (SEQ ID No. 10), and EIKDTKEAL (SEQ ID No. 692) may be used in a method for the diagnosis of an HIV infection.

The use of a ligand according to the invention, for the preparation of a vaccine intended for the prophylactic or therapeutic treatment of viral, bacterial, parasitic or fungal infections, is another subject of the invention.

The invention also relates to the use of a ligand according to the invention for the preparation of a vaccine intended for the prophylactic or therapeutic treatment of cancers, and preferably for inhibiting the growth of tumors.

The present invention also relates to the use of a physiologically acceptable strong acid for stabilizing and maintaining the biological activity of a molecule of pharmaceutical interest containing a glutamic acid or a glutamine at its N-terminal end.

In the preferred case where the molecule of pharmaceutical interest is an MHC ligand, the activity which it is sought to maintain is an activity of stimulation and of interaction with the cells of the immune system.

The invention also relates to the use of a strong acid for reducing and/or suppressing the formation of the pyroglutamic derivative of a molecule of pharmaceutical interest containing a glutamic acid or a glutamine at its N-terminal end.

Likewise, the present invention relates to a method for stabilizing a molecule of pharmaceutical interest containing a glutamic acid or a glutamine at its N-terminal end, characterized in that said molecule is reacted with a strong acid under conditions which make it possible to obtain said molecule in the form of a physiologically acceptable addition salt with a strong acid. The reaction with the strong acid is carried out in particular according to a method as defined below, it being possible for the strong acid to be chosen from the strong acids defined above, and makes it possible to obtain preferably a hydrochloride.

Indeed, the invention also relates to a method for preparing a molecule of pharmaceutical interest containing a glutamic acid or a glutamine at its N-terminal end in the form of a physiologically acceptable addition salt with a strong acid according to the invention.

This method may comprise in particular a step of purifying by RP-HPLC said molecule from the corresponding trifluoroacetate salt using an eluent based on said strong acid, optionally followed by a step of freeze-drying the solution thus obtained.

An alternative method comprises a step of dissolving a trifluoroacetate salt of said molecule in a solution of said strong acid in excess, optionally followed by a step of freeze-drying the solution thus obtained.

It is also possible to carry out the a method according to the invention which comprises an ion-exchange chromatography step starting with the corresponding trifluoroacetate salt of said molecule of pharmaceutical interest, after dissolving said salt in a solution containing said strong acid. The freeze-drying of the product obtained is also optional.

In all these applications, an MHC ligand, in particular SEQ ID No. 81, 112, 2, 273, 567, 10, 692, 11, 464, 466, 106, 257 or 568, is preferred. More preferably, it is SEQ ID No. 81 and the strong acid salt is a hydrochloride.

The examples which follow are intended to illustrate some embodiments of the invention and should not be considered as limiting the field of the invention.

### DESCRIPTION OF THE FIGURES

FIG. 1: Difference in cell lysis of the EL-4 A2/Kb cells prepulsed with the ELA peptide, by lymphocytes obtained after immunization of mice with the ELA (diamonds) or AcELA (squares) peptides in the presence of the adjuvant protein rP40, according to the protocol of example III.

FIG. 2: Generation of CTL after immunization, with the peptides ELA (trifluoroacetate, 2.A), ELA (hydrochloride, 2.B) or PyrELA (trifluoroacetate, 2.C) in the presence of the adjuvant protein rP40, according to the protocol of example IV.

FIG. 3: Chromatogram of the ELA peptide in acetate (3.A) or hydrochloride (3.B) form, stored at 37° C. for two months.

FIG. 4: Chromatogram of the ELA peptide in hydrochloride form initially (4.A) or after one month of storage at 4° C. (4.B).

### EXAMPLES

### Example I

### Synthesis of the Peptides ELA, PyrELA and ACELA

Peptide ELA: the peptide ELA (SEQ ID No. 81) is synthesized in a solid phase from the C-terminal amino acid toward the N-terminal amino acid (glutamic acid) in FMOC or tBOC chemistry. After cleavage of the resin and of the groups protecting the reactive side chains, the peptide is purified in a conventional manner with eluents based on trifluoroacetic acid/water and trifluoroacetic acid/acetonitrile before being freeze-dried. The purity of the peptide is checked by reversed-phase liquid chromatography. The amino acid composition is checked after hydrolysis and assay of the derived amino acids obtained. The exact mass is measured by mass spectrometry.

Peptide PyrELA: the peptide PyrELA is synthesized in the same manner as the peptide ELA, the only difference being the coupling of the last N-terminal amino acid: the glutamic acid is replaced by a pyroglutamic acid.

Peptide AcELA: the peptide AcELA is synthesized in the same manner as the peptide ELA, the only difference being a capping of the glutamic acid with the aid of acetic anhydride.

### Example II

### Preparation of a Hydrochloride Salt

II.A: Method A

Starting with the corresponding trifluoroacetate salt, a purification is carried out by RP-HPLC with the aid of an eluent A composed of water containing 0.1% HCl and an eluent B composed of 80% acetonitrile and 20% water containing 0.1% HCl.

A conventional freeze-drying step is then carried out.

II.B: Method B

Starting with the corresponding trifluoroacetate salt, dissolution is carried out in a solution with an excess of HCl and stirring is maintained for 2 hours. It is also possible to use an organic aqueous solution of the peptide in which HCl in gaseous form is bubbled.

A conventional freeze-drying step is then carried out.

II.C: Method C

his reaction is carried out starting with the corresponding trifluoroacetate salt, with the aid of an ion-exchange chromatography.

Commercially available ion-exchange resins in the hydrochloride form are used (Resin Dowex 1×4, Amberlite IRA 416), which can be used as such once regenerated.

a) Regeneration of the resin: the resin to be regenerated is introduced into a large column equipped with sintered glass of high porosity (1 or 2). The resin is then successively washed with ultra-pure water (pH 5-6), with 1N sodium hydroxide (pH 14), with ultra-pure water (pH 7), with 1N HCl (pH 1) and once again with ultra-pure water (pH 5-6). The resin is stored in an acetonitrile/10⁻⁴ N HCl (20/80) mixture at room temperature for at least one year.

b) Anion exchange (trifluoroacetate=>chloride): the peptide is dissolved in a 10⁻⁴ N HCl/acetonitrile solution, with a proportion of acetonitrile which may vary from 0 to 80%). The solution is injected at the top of the column. The peptide is eluted with the dissolution solution. The fractions containing the product are combined before being freeze-dried.

The quantity of hydrochloride may be assayed by an anion-exchange chromatography. The quantity of trifluoroacetic acid may be assayed by gas chromatography.

### Example III

### Generation of anti-Melan-A CTL after Immunization with rP40 Mixed with ELA or ACELA

Transgenic mice HLA-A* 0201/Kb (A2/Kb) of the strain C57B1/6×BDA/2 were used in this study (Vitiello et al., 1991, J. Exp. Med., 173, 1007). The MHC class I molecule expressed in these mice is a chimeric molecule formed of the a1 and a2 domains of the human molecule HLA-A0201 (allotype most frequently found) and of the a3 domain of the murine molecuke K^{b}.

A2/Kb mice received 300 μg of rP40 mixed with 50 μg of ELA or 300 μg of rP40 mixed with 50 μg of AcELA.

a) Generation of Effector Cytotoxic Cells:

10 days after immunization, the mice are sacrificed and the lymphocytes of the draining ganglia are recovered so as to be stimulated in vitro with the relevant peptide. These lymphocytes (4-5×10⁶) are cultured in a 24-well plate in DMEM plus 10 mM HEPES, 10% FCS and 50 μm β-2-mercaptoethanol with 2-5×10⁵ EL-4 A2/Kb cells (murine cells transfected with the HLA-A* 0201/Kb gene) which have been irradiated (10 kRads) and prepulsed for 1 h at 37° C. with 1 μM of the relevant peptide. After two weekly stimulations, the cells are tested for their cytotoxic activity.

b) Measurement of the Cytotoxic Activity:

The EL-4 A2/Kb cells are incubated for 1 h with ⁵¹Cr in the presence or otherwise of ELA, washed and then coincubated with the effector cells in various ratios in a 96-well plate in a volume of 200 μl for 4 to 6 h at 37° C. The cells are then centrifuged and the release of ⁵¹Cr is measured in 100 μl of supernatant. The percentage of specific lysis is calculated as follows:

% lysis=(experimental release−spontaneous release)/ (total release−spontaneous release)×100

% specific lysis=% lysis with cells pulsed with the peptide−% lysis with cells not pulsed with the peptide.

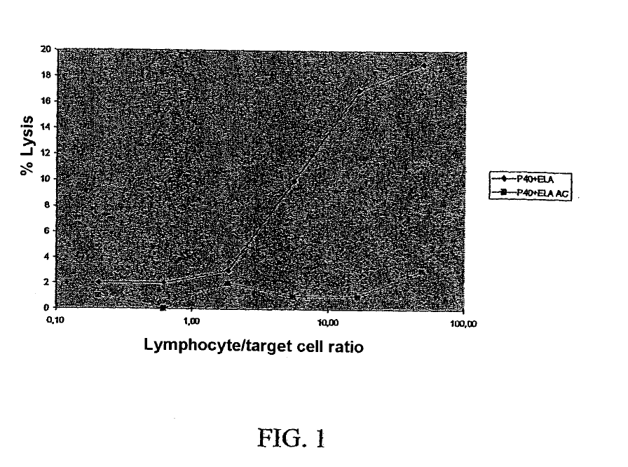

The difference in cell lysis observed for the ELA (diamonds) and AcELA (squares) peptides in the presence of the adjuvant protein rP40 (I. Rauly et al., Infect. Immun., 1999, 67, 5547) is represented in FIG. 1.

c) Conclusion:

Whereas an anti-ELA CTL activity is observed after immunization of mice with P40/ELA, no CTL activity is measured when the mice were immunized with P40/AcELA. These results indicate that the CTLs generated by AcELA do not recognize the native ELA peptide.

### Comparative Example IV

### CTL Activity of the Peptides ELA, PyrELA and ACELA

A2/Kb mice received:

300 μg of rP40 mixed with 50 μg of ELA (Trifluoroacetate)

300 μg of rP40 mixed with 50 μg of ELA (Hydrochloride)

300 μg of rP40 mixed with 50 μg of PyrELA (Trifluoroacetate)

a) Generation of Effector Cytotoxic Cells:

10 days after immunization, the mice are sacrificed and the lymphocytes of the draining ganglia are recovered so as to be stimulated in vitro with the relevant peptide. These lymphocytes (4-5×10⁶ ) are cultured in a 24-well plate in DMEM plus 10 mM HEPES, 10% FCS and 50 μm β-2-mercaptoethanol with 2-5×10⁵ EL-4 A2/Kb cells (murine cells transfected with the HLA-A* 0201/Kb gene) which have been irradiated (10 kRads) and prepulsed for 1 h at 37° C. with 1 μM of the relevant peptide. After two weekly stimulations, the cells are tested for their cytotoxic activity.

b) Measurement of the Cytotoxic Activity:

The EL-4 A2/Kb cells are incubated for 1 h with ⁵¹Cr in the presence or otherwise of ELA, washed and then coincubated with the effector cells in various ratios in a 96-well plate in a volume of 200 μl for 4 to 6 h at 37° C. The cells are then centrifuged and the release of ⁵¹Cr is measured in 100 μl of supernatant. The percentage of specific lysis is calculated as follows:

% lysis=(experimental release−spontaneous release)/ (total release−spontaneous release)×100

% specific lysis=% lysis with cells pulsed with the peptide−% lysis with cells not pulsed with the ELA peptide.

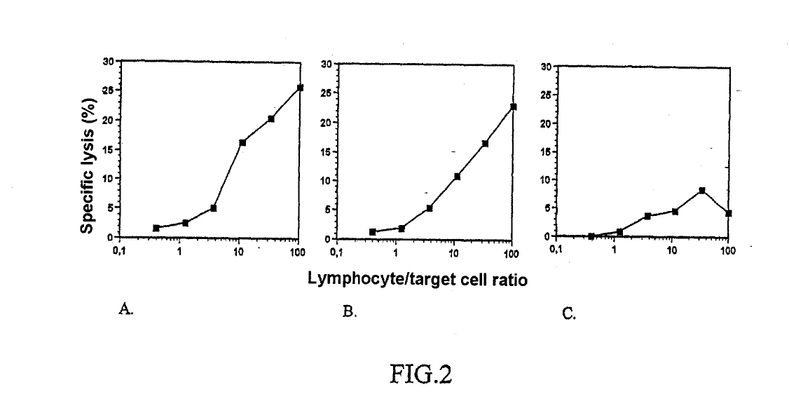

c) The Generation of Anti-Melan-A CTL after Immunization with rP40 mixed with the Peptides ELA (Trifluoroacetate), ELA (Hydrochloride) or PyrELA (Trifluoroacetate) is represented in FIG. 2.

d) Conclusions:

1. Whereas an anti-ELA CTL activity is observed after immunization of mice with P40/ELA (Trifluoroacetate), no CTL activity is measured when the mice were immunized with P40/PyrELA (Trifluoroacetate). These results indicate that the CTLs generated by PyrELA do not recognize the native ELA peptide.

2. Surprisingly, the immunization with P40/ELA (Hydrochloride) is as effective as that with P40/ELA (Trifluoroacetate) for generating an anti-ELA CTL response.

### Example V

### Studies of Accelerated Stability of the Acetate and Hydrochloride Forms of the ELA Peptide

The peptides are analyzed by reversed-phase HPLC with the aid of an eluent A composed of water containing 0.1% TFA and an eluent B composed of 80% acetonitrile and 20% water containing 0.1% TFA.

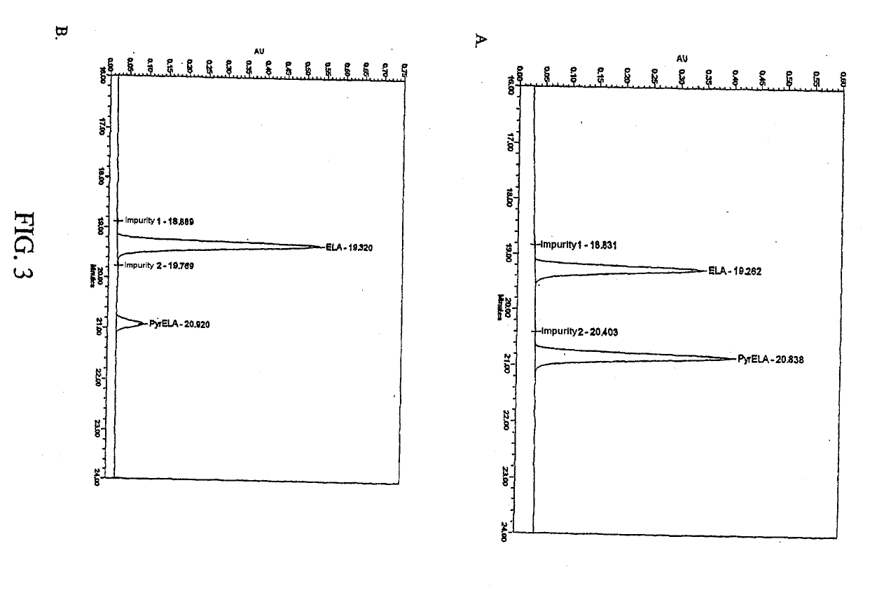

FIG. 3 shows the chromatograms for the ELA peptide in acetate (3.A) or hydrochloride (3.B) form stored at 37° C. for 2 months.

Conclusion:

In the acetate form, the degradation of the ELA peptide to an inactive cyclized peptide PyrELA after 2 months at 37° C. is 53%. Surprisingly, in the hydrochloride form, it is only 10%.

### Example VI

### Stability of the ELA Peptide in the Hydrochloride Form Stored at 4° C.

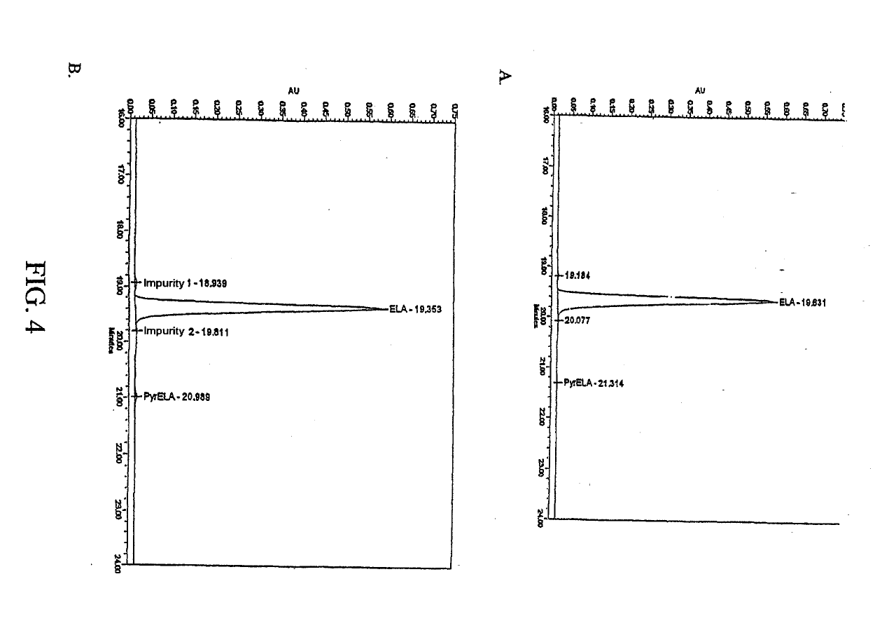

FIG. 4 shows a chromatogram for the ELA peptide in the hydrochloride form at t=0: (98.9% of ELA and 0.4% of PyrELA; FIG. 4.A) and after one month of storage at 4° C. (98.8% of ELA and 0.5% of PyrELA; FIG. 4.B).

Conclusion:

Surprisingly, the ELA peptide in the hydrochloride form is extremely stable at 4° C. It can therefore be easily handled and stored at a temperature of 4 or −20° C. That is not the case for an equivalent peptide (MART 3), prepared in the acetate form which must be stored at −80° C. (M. Marchand et al., Int. J. Cancer, 1999, 80, 219).

The strong acid saline form therefore allows a much easier storage at 4° C. (refrigerator) or at −20° C. (freezer) with total physicochemical stability, as shown by the examples above.

1. A molecule of pharmaceutical interest containing at its N-terminal end a glutamic acid or a glutamine, characterized in that it exists in the form of a physiologically acceptable addition salt with a strong acid.

2. The molecule of pharmaceutical interest as claimed in claim 1, characterized in that it is an MHC ligand containing at its N-terminal end a glutamic acid or a glutamine.

3. The molecule of pharmaceutical interest as claimed in claim 1 or 2, characterized in that the physiologically acceptable addition salt with a strong acid is chosen from the addition salts with inorganic or organic acids preferably from the methanesulfonate, hydrochloride, hydrobromide, sulfate, nitrate and phosphate.

4. The molecule of pharmaceutical interest as claimed in one of claims 1 to 3, characterized in that it is chosen from natural or synthetic molecules.

5. The molecule of pharmaceutical interest as claimed in one of claims 1 to 4, characterized in that it is chosen from the group consisting of proteins, peptides, multi-epitope polypeptide constructs, pseudopeptides, retro-inverso, peptoids, peptidomimetics and lipopeptides.

6. The MHC ligand as claimed in one of claims 2 to 4, characterized in that it is chosen from the CTL epitopes.

7. The MHC ligand as claimed in claim 6, characterized in that it is chosen from the CTL Epitopes existing in the form of an octapeptide, nonapeptide or decapeptide.

8. The MHC ligand as claimed in one of claims 2 to 4, characterized in that it is chosen from the ligands described in the databases SYFPEITHI or MHCPEP containing a glutamic acid or a glutamine at their N-terminal end.

9. The MHC ligand as claimed in either of claims 2 and 3, characterized in that it is chosen from the peptides SEQ ID No. 1 to SEQ ID No. 695.

10. The MHC ligand as claimed in one of claims 2 to 7, characterized in that it is chosen from the group of peptides corresponding to SEQ ID No. 81, SEQ ID No. 112, SEQ ID No. 2, SEQ ID No. 273, SEQ ID No. 110, SEQ ID No. 106, SEQ ID No. 10, SEQ ID No. 692, SEQ ID No. 257, SEQ ID No. 568, SEQ ID No. 464, SEQ ID No. 466, SEQ ID No. 567 and SEQ ID No. 695.

11. The MHC ligand as claimed in claim 10, characterized in that it is the peptide corresponding to SEQ ID No. 81, in hydrochloride or sulfate form.

12. A pharmaceutical composition, characterized in that it comprises at least one molecule of pharmaceutical interest as claimed in one of claims 1 to 11.

13. A vaccine, characterized in that it comprises at least one MHC ligand as claimed in one of claims 2 to 11.

14. The vaccine as claimed in claim 13, characterized in that it comprises, in addition, at least one adjuvant.

15. The vaccine as claimed in claim 13 or 14, characterized in that the adjuvant is chosen from the salts of Aluminum (Alum) or of Calcium, the enterobacterial OmpA proteins, TT, DT, CRM197, PLGA, ISCOM, Montanide ISA 720, aliphatic quaternary ammoniums, MPL-A, Quil-A, CpGs, Leif, CT, LT or the detoxified versions of CT or LT.

16. The vaccine as claimed in one of claims 13 to 15, characterized in that it comprises, in addition, a carrier compound mixed with or coupled to said ligand.

17. The vaccine as claimed in claim 15, characterized in that said carrier compound is chosen from toxoids, including the diphtheria toxoid or the tetanus toxoid, proteins derived from streptococcus, bacterial outer membrane proteins of the OmpA type, outer membrane protein complexes (OMPC), outer membrane vesicles (OMV) or HSPs.

18. The vaccine as claimed in one of claims 13 to 17, characterized in that said ligand optionally combined with a carrier compound is incorporated into a vector chosen from the group comprising liposomes, virosomes, nanospheres, microspheres, microcapsules or biovectors.

19. An anti-melanoma vaccine, characterized in that it comprises at least one peptide as claimed in claim 11.

20. The anti-melanoma vaccine as claimed in claim 19, characterized in that it comprises, in addition, an enterobacterial OmpA protein.

21. A method for the in vitro diagnosis of pathologies associated with the presence, in a patient's body, of MHC ligands, and which may be directly or indirectly involved in the process of development of these pathologies in humans or , animals, characterized in that it comprises the steps of:
bringing a biological sample obtained from a patient, in particular blood or any biological sample which may contain lymphocytes, into contact with an MHC ligand according to the invention, under conditions allowing the formation of a binary complex between said MHC ligand and the MHC molecules present in said sample, and the reaction between said binary complex and the T cell receptors which may be present in said biological sample,
detecting in vitro the ternary complex MHC—MHC ligand—T receptor, which may be formed in the preceding step.

22. A pack or kit for carrying out diagnostic methods in vitro as claimed in claim 21, comprising:
an MHC ligand according to one of claims 2 to 11;
optionally reagents to allow the formation of an immunological reaction between said ligand, the MHC molecules and the T cell receptors which may be present in the biological sample;
optionally reagents which make it possible to detect the ternary complex according to the invention, which was produced at the end of the immunological reaction, said reagents optionally containing a marker or being capable of being recognized in turn by a labeled reagent.

23. The use of a ligand as claimed in one of claims 2 to 10, for the preparation of a vaccine intended for the prophylactic or therapeutic treatment of viral, bacterial, parasitic or fungal infections.

24. The use of a ligand as claimed in one of claims 2 to 11, for the preparation of a vaccine intended for the prophylactic or therapeutic treatment of cancers, and preferably for inhibiting the growth of tumors.

25. The use of a physiologically acceptable strong acid for stabilizing and maintaining the biological activity of a molecule with pharmaceutical activity containing a glutamic acid or a glutamine at its N-terminal end.

26. The use of a strong acid for reducing and/or suppressing the formation of the pyroglutamic derivative of a molecule with pharmaceutical activity containing a glutamic acid or a glutamine at its N-terminal end.

27. A method for preparing a molecule of pharmaceutical interest containing a glutamic acid or a glutamine at its N-terminal end in the form of a physiologically acceptable addition salt with a strong acid as claimed in one of claims 1 to 11, characterized in that it comprises a step of purifying by RP-HPLC said molecule from the corresponding trifluoroacetate salt using an eluent based on said strong acid, optionally followed by a step of freeze-drying the solution thus obtained.

28. A method for preparing a molecule of pharmaceutical interest containing a glutamic acid or a glutamine at its N-terminal end in the form of a physiologically acceptable addition salt with a strong acid as claimed in one of claims 1 to 11, characterized in that it comprises a step of dissolving a trifluoroacetate salt of said molecule in a solution of said strong acid in excess, optionally followed by a step of freeze-drying the solution thus obtained.

29. A method for preparing a molecule of pharmaceutical interest containing a glutamic acid or a glutamine at its N-terminal end in the form of a physiologically acceptable addition salt with a strong acid as claimed in one of claims 1 to 11, characterized in that it comprises an ion-exchange chromatography step starting with the corresponding trifluoroacetate salt of said molecule of pharmaceutical interest, after dissolving said salt in a solution containing said strong acid.

30. A method for stabilizing a molecule of pharmaceutical interest containing a glutamic acid or a glutamine at its N-terminal end, characterized in that said molecule is reacted with a strong acid under conditions which make it possible to obtain said molecule in the form of a physiologically acceptable addition salt with a strong acid, in particular according to a method as claimed in one of claims 27 to 29.
